(19) United States
(12) Patent Application Publication
(10) Pub. No.: US 2026/0355297 A1
(43) Pub. Date: Nov. 12, 2026

(54) TREATMENT OF TYPE 1 DIABETES (T1D) WITH TOLERIZING NANOPARTICLES

(71) Applicant: COUR PHARMACEUTICALS DEVELOPMENT COMPANY INC., Evanston, IL (US)

(72) Inventors: Adam Elhofy, Chicago, IL (US); Michael Boyne, Skokie, IL (US); Greta Wodarcyk, Northbrook, IL (US)

(21) Appl. No.: 19/485,827

(22) PCT Filed: May 24, 2024
(86) PCT No.: PCT/US2024/031020

§ 371 Date: Nov. 19, 2025

Related U.S. Application Data
(60) Provisional application No. 63/504,271, filed on May 25, 2023.
(60) Provisional application No. 63/566,571, filed on Mar. 18, 2024.

(51) Int. Cl. A61K 39/00; A61K 9/51; A61K 38/28; A61P 3/10; A61P 37/06

(52) U.S. Cl. CPC A61K 39/0008; A61K 9/51; A61K 38/28; A61P 3/10; A61P 37/06; A61K 2039/54; A61K 2039/545

(57) ABSTRACT

The present disclosure relates to methods of treating Type 1 diabetes (T1D) using tolerizing immune modifying nanoparticles encapsulating T1D associated antigens.

### CROSS REFERENCE TO RELATED APPLICATIONS

The present application claims the priority benefit of U.S. Provisional Patent Application No. 63/504,271, filed May 25, 2023, and U.S. Provisional Patent Application No. 63/566,571, filed Mar. 18, 2024, each of which is incorporated by reference here in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: filename: 59248_seqlisting.xml; size: 23,766 bytes; created: May 24, 2024.

### FIELD OF THE DISCLOSURE

The present disclosure relates to methods of treating Type 1 Diabetes (T1D) using tolerizing immune modifying nanoparticles encapsulating T1D associated antigens.

### BACKGROUND

Type 1 diabetes (T1D), also referred to as type 1 diabetes mellitus (T1DM), is an autoimmune disease characterized by T and B cell responses to proteins expressed by insulin-secreting pancreatic β-cells, inflammatory lesions within islets (insulitis), and β-cell loss.

T1D affects ~1.6 million people in the United States and ~4.7 million people world-wide¹. The incidence of T1D is highest in children although T1D onset can occur at any age. There appears to be no sex difference in the overall incidence of childhood diabetes with the same male to female ratio².

Autoimmune activity in T1D is targeted primarily to β-cell proteins. Inflammatory lesions within pancreatic islets results in patients' inability to effectively stabilize adequate glucose metabolic control.

The burden of T1D is vast and yet there is no cure for T1D. The current standard of treatment for T1D relies on exogenous insulin replacement to manage blood sugar levels combined with diet and exercise modifications. The newly approved drug teplizumab can delay the onset of T1D by an average of 2 years, but neither cures nor treats the root cause of T1D, that is activated autoimmune T cells mediating continued pancreatic βcell destruction through CD8⁺ T cells, CD4⁺ T cell mediated inflammation, or autoantibodies.

### SUMMARY

Tolerizing immune modifying particles (TIMPs), comprising one or more antigens, have been previously described for the treatment of immune-mediated disorders (e.g autoimmune diseases and allergics) via induction of antigen-specific immune tolerance (WO2013192532 and WO2015023796 incorporated herein by reference). In several preclinical models of autoimmune diseases and allergies, TIMPs have demonstrated efficacy at inducing T-cell tolerance. Induction of antigen-specific tolerance to T1D autoantigens using TIMPs encapsulating T1D-associated antigens (TIMP-T1D) could ameliorate or potentially cure T1D.

Provided herein is a method of treating T1D in a subject in need thereof comprising administering to the subject TIMP-T1D, wherein TIMP-T1D is administered at a dose determined based on the subject's weight. In various embodiments, TIMP-T1D is administered at a dose of 0.01 to 12 mg/kg inclusive of all values and ranges between these values. In various embodiments, TIMP-T1D is administered at a fixed dose between 1 mg to 800 mg inclusive of all values and ranges between these values. In various embodiments, the disclosure provides a method of treating Type 1 diabetes (T1D) in a subject comprising administering to the subject tolerizing immune modifying particles encapsulating one or more T1D associated antigens (TIMP-T1D), wherein TIMP-T1D is administered at a dose level between 1 mg and 800 mg. In various embodiments, the TIMP-T1D is administered at a dose from about 0.01 to about 12 mg/kg, from about 0.05 to about 10 mg/kg, from about 0.01 to about 5 mg/kg, from about 0.1 to about 10 mg/kg, from about 1 to about 8 mg/kg, from about 1.5 to about 10 mg/kg, from about 2 to about 12 mg/kg, from about 2 to about 10 mg/kg, from about 3 to about 10 mg/kg, from about 4 to about 10 mg/kg, from about 4 to about 12 mg/kg, or from about 5 to about 12 mg/kg. In various embodiments, TIMP-T1D is administered at a dose of about 0.01 mg/kg, about 0.05 mg/kg, about 0.1 mg/kg, about 0.25 mg/kg, about 0.5 mg/kg, about 1.0 mg/kg, about 2.0 mg/kg, about 3.0 mg/kg, about 4.0 mg/kg, about 5 mg/kg, about 6 mg/kg, about 8.0 mg/kg, about 10 mg/kg, or about 12 mg/kg. In various embodiments, TIMP-T1D is administered at a fixed dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, the method further comprises administering at least one booster dose of TIMP-T1D. In various embodiments, a booster dose of TIMP-T1D is administered in a single dose or in multiple doses following the loading TIMP-T1D administration. In various embodiments, the booster dose is administered once weekly, once every two weeks, once every three weeks, once every 4 weeks, once every two months, once every three months, once every 4 months, once every 5 months, once every 6 months, or once per year. In various embodiments, TIMP-T1D is administered in two loading doses one-week apart followed by at least one booster dose administered as a single dose once every one month, every two months or three months. In various embodiments, TIMP-T1D is administered in two loading doses one-week apart followed by at least one booster dose administered as a single dose once every one month, every two months or three months, once every 4 months, once every 5 months, once every 6 months, or once per year. In various embodiments, TIMP-T1D is administered in two loading doses one-week apart followed by a booster dose administered as a single dose once every three months. In various embodiments, one, two, three, four or five booster doses are administered to a subject.

In various embodiments, booster dose of TIMP-T1D is administered at a dose of 0.01 to 12 mg/kg inclusive of all values and ranges between these values. In various embodiments, the booster dose of TIMP-T1D is administered at a dose from about 0.01 to about 12 mg/kg, from about 0.05 to about 10 mg/kg, from about 0.01 to about 5 mg/kg, from about 0.1 to about 10 mg/kg, from about 1 to about 8 mg/kg, from about 1.5 to about 10 mg/kg, from about 2 to about 12 mg/kg, from about 2 to about 10 mg/kg, from about 3 to about 10 mg/kg, from about 4 to about 10 mg/kg, from about 4 to about 12 mg/kg, or from about 5 to about 12 mg/kg, or about 0.01 mg/kg, about 0.05 mg/kg, about 0.1 mg/kg, about 0.25 mg/kg, about 0.5 mg/kg, about 1.0 mg/kg, about 2.0 mg/kg, about 3.0 mg/kg, about 4.0 mg/kg, about 5 mg/kg, about 6 mg/kg, about 8.0 mg/kg, about 10 mg/kg, or about 12 mg/kg. In various embodiments, the booster dose of TIMP-T1D is administered at a dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, the subject in need thereof has been diagnosed with T1D within 1 week to within 7 years prior to administering TIMP-T1D. In various embodiments, the subject in need thereof has been diagnosed within 1 week, 2 weeks, 3 weeks, 4 weeks, 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, or 7 years including all ranges and values lying within the range.

In various embodiments, the subject in need thereof has recent-onset T1D. In various embodiments, the subject in need thereof has stage 3 T1D. In various embodiments, the subject in need thereof has ≥1 auto-antibody specific for pancreatic β-cell proteins, GAD65, IA-2, ZnT8, insulin, or islet cell cytoplasmic autoantibodies. In various embodiments, the subject in need thereof has an autoantibody level <50,000 IU/mL. In various embodiments, the subject in need thereof has a fasting plasma glucose (FPG) level ≥126 mg/dL (7.0 mmol/L). In various embodiments, the subject in need thereof has a random plasma glucose ≥200 mg/dl (11.1 mmol/L) with classic symptoms of hyperglycemia or hyperglycemic crisis. In various embodiments, the subject in need thereof has peak stimulated C-peptide of ≥0.2 nmol/L during a mixed meal tolerance test. In various embodiments, the subject in need thereof has a fasting C-peptide level of ≥0.12 nmol/L (≥0.36 ng/mL). In certain embodiments, the subject in need thereof has a fasting C-peptide level ≥0.12-≤1.5 nmol/L (≥0.36-≤4.5 ng/mL). In certain embodiments, the subject in need thereof possesses HbA1c levels ≥5.6%. In various embodiments, the subject in need thereof possesses HbA1c levels between about 5.6% to about 9.5% (about 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, or 9.5% including all ranges and values lying within that range. In certain embodiments, the subject in need thereof possesses HbA1c levels about ≤12%. In various embodiments, the subject in need thereof is on a stable insulin dose or insulin dosing regimen between 1 week and 1 month (1 week, 2 weeks, 3 weeks, 4 weeks including values lying within that range). In various embodiments, the subject in need thereof has at least 20% of beta-cell function prior to administration of the first dose. In various embodiments, the subject in need thereof is between about 7 and 75 years of age (about 7, 8, 9, 10, 11, 12, 13 . . . 72, 73, 74, or 75 years of age including all ranges and values lying within that range).

In various embodiments, administering the booster dose is determined based on one or more of the following assessments: Change in area under the curve (AUC) of stimulated C-peptide response over the first 2 hours of a 2 or 4-hour MMTT, Maintenance of C-peptide levels, Change in maximum C-peptide during MMTT, Change in C-peptide measured at 30, 60, 90, and 120 minutes during MMTT, Change in fasting C-peptide, Proinsulin-to-C-peptide ratio, Change in antigen specific T cells, Change in activated antigen specific T cells, Change in antigen specific Tregs, Change in HbA1c levels, Change in number of hypoglycemic or severe hypoglycemic events, Change in rate of hypoglycemic events, Change in exogenous insulin use/consumption, Change in time in glycemic target range of 70-180 mg/dL (3.9-10.0 mmol/L), Change in time in glycemic target range of 70-110 mg/dL (3.9-6.1 mmol/L), Change in insulin-dose-adjusted HbA1c (IDAA1c), Insulin sensitivity score (ISS), Change in glycemic variability/fluctuations, Proportion of patients with IDAA1c≤9, Proportion of patients with a stimulated maximum C-peptide level above 0.2 nmol/L (0.6 ng/ml) at 15 months, Proportion of patients with a stimulated 90 min C-peptide level above 0.2 nmol/L (0.6 ng/ml) at 15 months, Change in body weight and body mass index, Concentrations of serum autoantibodies (number, and titer), Concentrations of serum autoantibody isotypes (IgG1, IgG2, IgG3, IgG4), Secretion of cytokines IL-1, IL-2, IL-5, IL-13, IL-10, IL-17, interferon (IFN) γ, tumor necrosis factor (TNF)α by PBMCs upon stimulation with T1D-associated antigens, Serum concentrations of cytokines IL-1, IL-2, IL-5, IL-13, IL-10, IL-17, IFNγ, TNFα, Secretion of cytokines IL-1, IL-2, IL-5, IL-13, IL-10, IL-17, IFNγ, TNFα by PBMCs upon stimulation with anti-CD3 and anti-CD28, Proliferation of PBMCs upon stimulation with T1D-associated antigens, Circulating methylated- and unmethylated-insulin DNA levels, Circulating hormone levels (e.g. glucagon, incretins, adiponectin).

In various embodiments, the subject in need thereof is between about 7 and 17 years of age. In various embodiments, the subject in need thereof is between about 12 and about 35 years of age. In various embodiments, the subject in need thereof is between about 18 and about 35 years of age including all ranges and values lying within that range. In various embodiments, the subject in need thereof is between about 18 and 75 years of age. In various embodiments, a subject in need thereof has an HLA genotype selected from the group consisting of HLA-DQ, HLA-DR, HLA-DP, or HLA-B. In various embodiments, a subject in need thereof has an HLA genotype selected from the group consisting of DR3, DR4, or DQ8. In various embodiments, a subject in need thereof has an HLA genotype selected from the group consisting of DRB/*03:01-DQA1*05:01-DQB1*02:01 (abbreviated “DR3”), DRB1*04:01/02/047/05/08-DQA1*03:01-DQB1*03:02/04 (or DQB1*02; abbreviated “DR4”), DR3-DR4, DPB1*03:01, DPB1*02:02, B*39:06, A*02:01, A*24:02, B*18:01, or C*03:03.

In various embodiments, TIMP-T1D encapsulates one or more T1D-associated antigens or antigenic epitopes. In various embodiments, the T1D-associated antigens are selected from the group consisting of insulin (preproinsulin (PPI)), tyrosine phosphatase like autoantigen or insulinoma antigen-2 (IA2; ICA512, PTPRN), IA-2β (Phogrin, PTPRN2), glutamic acid decarboxylase 65-kDa isoform (GAD65), glutamic acid decarboxylase 67-kDa isoform (GAD67), islet cell antigen-69 (ICA69), Islet-specific glucose-6-phosphatase catalytic subunit-related protein (IGRP), Zinc transporter 8 (ZnT8), Chromogranin A, 38 kDa granule antigen, peripherin, Islet amyloid peptide (ppIAPP), carboxypeptidase H/E, Heat shock protein 60 (HSP60), Heat shock protein 70 family (e.g., HSPA1A, HSPA1B, HSPA1L, HSPA2, HSPA5, HSPA6, HSPA8, HSPA9, and other family members) sulfatide, or vasostatin-1.

In various embodiments, the T1D-associated antigens are set out in Table 1. In various embodiments, the T1D-associated antigens are set out in Table 2. In various embodiments, the T1D associated antigen is selected from the group consisting of SEQ ID NO: 1 to SEQ ID NO: 17. In various embodiments, TIMP-T1D encapsulates T1D-associated antigens comprising PPI, ZnT8, GAD65, and IGRP.

In various embodiments, TIMP-T1D encapsulates T1D-associated antigens comprising SEQ ID NO 1, SEQ ID NO 2, SEQ ID NO 3, and SEQ ID NO 4. In various embodiments, TIMP-T1D encapsulates T1D-associated antigens comprising SEQ ID NO: 1, SEQ ID NO: 2, SEQ ID NO: 3, and SEQ ID NO: 16. In various embodiments, the ZnT8 protein lacks the last amino acid, aspartic acid (D), in SEQ ID NO: 4, and has the sequence set out in SEQ ID NO: 16 (ZnT8 variant).

TABLE 1

| Protein name | UNIPROT ID |
|---|---|
| Insulin (preproinsulin (PPI)) | P0138 |
| Tyrosine phosphatase like autoantigen or | Q16849 |
| insulinoma antigen-2 (IA2; ICA512, PTPRN) | |
| IA-2β (Phogrin, PTPRN2) | Q92932 |
| Glutamic acid decarboxylase 65-kDa isoform | Q05329, Q9UGI5 |
| (GAD65) | |
| glutamic acid decarboxylase 67-kDa isoform | Q99259 |
| (GAD67) | |
| Islet cell antigen-69 (ICA69) | Q05084, Q6LCT9 |
| Islet-specific glucose-6-phosphatase catalytic | Q9NQR9 |
| subunit-related protein (IGRP) | |
| Zinc transporter 8 (ZnT8) | Q8IWU4 |
| Chromogranin A | P10645, G5E968, G3V2Q7 |
| Peripherin | P41219, P23942 |
| Islet amyloid peptide (ppIAPP) | P10997 |
| carboxypeptidase H/E | P16870 |
| Heat shock protein 60 (Hsp60) | P10809 |
| Heat shock protein 70 (Hsp70) family | P0DMV9, P0DMV8, |
| | P17066, P54652, |
| | P11021, P11142, |
| | P38646, P34931 |

TABLE 2

| | SEQ | |
|---|---|---|
| | ID | |
| Protein name | NO | Sequence |
| Insulin | 1 | MALWMRLLPLLALLALWGPDPAAAFVNOHLCGSHLVEALYLVCGERGFFYTP |
| (preproinsulin (PPI)) | | KTRREAEDLQVGQVELGGGPGAGSLQPLALEGSLOKRGIVEQCCTSICSLYQ |
| | | LENYCN |
| Glutamic acid | 2 | MNYAFLHATDLLPACDGERPTLAFLODVMNILLQYVVKSFDRSTKVIDFHYP |
| decarboxylase 65- | | NELLQEYNWELADQPQNLEEILMHCQTTLKYAIKTGHPRYFNQLSTGLDMVG |
| kDa isoform | | LAADWLTSTANTNMFTYEIAPVFVLLEYVTLKKMREIIGWPGGSGDGIFSPG |
| (GAD65) | | GAISNMYAMMIARFKMFPEVKEKGMAALPRLIAFTSEHSHFSLKKGAAALGI |
| | | GTDSVILIKCDERGKMIPSDLERRILEAKQKGFVPFLVSATAGTTVYGAFDP |
| | | LLAVADICKKYKIWMHVDAAWGGGLLMSRKHKWKLSGVERANSVTWNPHKMM |
| | | GVPLQCSALLVREEGLMQNCNQMHASYLFQQDKHYDLSYDTGDKALQCGRHV |
| | | DVFKLWLMWRAKGTTGFEAHVDKCLELAEYLYNIIKNREGYEMVFDGKPQHT |
| | | NVCFWYIPPSLRTLEDNEERMSRLSKVAPVIKARMMEYGTTMVSYQPLGDKV |
| | | NFFRMVISNPAATHQDIDFLIEEIERLGQDL |
| Islet-specific | 3 | MDFLHRNGVLIIQHLQKDYRAYYTFLNFMSNVGDPRNIFFIYFPLCFQFNQT |
| glucose-6- | | VGTKMIWVAVIGDWLNLIFKWILFGHRPYWWVQETQIYPNHSSPCLEQFPTT |
| phosphatase | | CETGPGSPSGHAMGASCVWYVMVTAALSHTVCGMDKFSITLHRLTW |
| catalytic subunit- | | SFLWSVFWLIQISVCISRVFIATHFPHQVILGVIGGMLVAEAFEHTPGIQTA |
| related protein | | SLGTYLKTNLFLFLFAVGFYLLLRVLNIDLLWSVPIAKKWCANPDWIHIDTT |
| (IGRP) | | PFAGLVRNLGVLFGLGFAINSEMFLLSCRGGNNYTLSFRLLCALTSLTILQL |
| | | YHFLQIPTHEEHLFYVLSFCKSASIPLTVVAFIPYSVHMLMKQSGKKSQ |
| Zinc transporter 8 | 4 | MEFLERTYLVNDKAAKMYAFTLESVELQQKPVNKDQCPRERPEELESGGMYH |
| (ZnT8) | | CHSGSKPTEKGANEYAYAKWKLCSASAICFIFMIAEVVGGHIAGSLAVVTDA |
| | | AHLLIDLTSFLLSLFSLWLSSKPPSKRLTFGWHRAEILGALLSILCIWVVTG |
| | | VLVYLACERLLYPDYQIQATVMIIVSSCAVAANIVLTVVLHQRCLGHNHKEV |
| | | QANASVRAAFVHALGDLFQSISVLISALIIYFKPEYKIADPICTFIFSILVL |
| | | ASTITILKDFSILLMEGVPKSLNYSGVKELILAVDGVLSVHSLHIWSLTMNQ |
| | | VILSAHVATAASRDSQVVRREIAKALSKSFTMHSLTIQ MESPVDQDPD |
| | | CLFCEDPCD |
| Tyrosine | 5 | MRRPRRPGGLGGSGGLRLLLCLLLLSSRPGGCSAVSAHGCLFDRRLCSHLEV |
| phosphatase like | | CIQDGLFGQCQVGVGQARPLLQVTSPVLQRLQGVLRQLMSQGLSWHDDLTQY |
| autoantigen or | | VISQEMERIPRLRPPEPRPRDRSGLAPKRPGPAGELLLQDIPTGSAPAAQHR |
| insulinoma antigen-2 | | LPQPPVGKGGAGASSSLSPLQAELLPPLLEHLLLPPQPPHPSLSYEPALLQP |
| (IA2; ICA512, PTPRN) | | YLFHQFGSRDGSRVSEGSPGMVSVGPLPKAEAPALFSRTASKGIFGDHPGHS |
| | | YGDLPGPSPAQLFQDSGLLYLAQELPAPSRARVPRLPEQGSSSRAEDSPEGY |
| | | EKEGLGDRGEKPASPAVQPDAALQRLAAVLAGYGVELRQLTPEQLSTLLTLL |
| | | QLLPKGAGRNPGGVVNVGADIKKTMEGPVEGRDTAELPARTSPMPGHPTASP |
| | | TSSEVQQVPSPVSSEPPKAARPPVTPVLLEKKSPLGQSQPTVAGQPSARPAA |
| | | EEYGYIVTDQKPLSLAAGVKLLEILAEHVHMSSGSFINISVVGPALTFRIRH |
| | | NEQNLSLADVTQQAGLVKSELEAQTGLQILQTGVGQREEAAAVLPQTAHSTS |
| | | PMRSVLLTLVALAGVAGLLVALAVALCVRQHARQQDKERLAALGPEGAHGDT |
| | | TFEYQDLCRQHMATKSLFNRAEGPPEPSRVSSVSSQFSDAAQASPSSHSSTP |
| | | SWCEEPAQANMDISTGHMILAYMEDHLRNRDRLAKEWQALCAYQAEPNTCAT |
| | | AQGEGNIKKNRHPDFLPYDHARIKLKVESSPSRSDYINASPIIEHDPRMPAY |
| | | IATQGPLSHTIADFWQMVWESGCTVIVMLTPLVEDGVKQCDRYWPDEGASLY |
| | | HVYEVNLVSEHIWCEDFLVRSFYLKNVQTQETRTLTQFHFLSWPAEGTPAST |
| | | RPLLDFRRKVNKCYRGRSCPIIVHCSDGAGRTGTYILIDMVLNRMAKGVKEI |
| | | DIAATLEHVRDQRPGLVRSKDQFEFALTAVAEEVNAILKALPQ |
| IA-2β (Phogrin, | 6 | MGPPLPLLLLLLLLLPPRVLPAAPSSVPRGRQLPGRLGCLLEEGLCGASEAC |
| PTPRN2) | | VNDGVFGRCQKVPAMDFYRYEVSPVALQRLRVALQKLSGTGFTWQDDYTQYV |
| | | MDQELADLPKTYLRRPEASSPARPSKHSVGSERRYSREGGAALANALRRHLP |
| | | FLEALSQAPASDVLARTHTAQDRPPAEGDDRFSESILTYVAHTSALTYPPGS |
| | | RTQLREDLLPRTLGQLQPDELSPKVDSGVDRHHLMAALSAYAAQRPPAPPGE |
| | | GSLEPQYLLRAPSRMPRPLLAPAAPQKWPSPLGDSEDPSSTGDGARIHTLLK |
| | | DLQRQPAEVRGLSGLELDGMAELMAGLMQGVDHGVARGSPGRAALGESGEQA |
| | | DGPKATLRGDSFPDDGVQDDDDRLYQEVHRLSATLGGLLQDHGSRLLPGALP |
| | | FARPLDMERKKSEHPESSLSSEEETAGVENVKSQTYSKDLLGQQPHSEPGAA |
| | | AFGELQNQMPGPSKEEQSLPAGAQEALSDGLQLEVQPSEEEARGYIVTDRDP |
| | | LRPEEGRRLVEDVARLLQVPSSAFADVEVLGPAVTFKVSANVQNVTTEDVEK |
| | | ATVDNKDKLEETSGLKILQTGVGSKSKLKFLPPQAEQEDSTKFIALTLVSLA |
| | | CILGVLLASGLIYCLRHSSQHRLKEKLSGLGGDPGADATAAYQELCRQRMAT |
| | | RPPDRPEGPHTSRISSVSSQFSDGPIPSPSARSSASSWSEEPVQSNMDISTG |
| | | HMILSYMEDHLKNKNRLEKEWEALCAYQAEPNSSEVAQREENVPKNRSLAVL |
| | | TYDHSRVLLKAENSHSHSDYINASPIMDHDPRNPAYIATQGPLPATVADFWQ |
| | | MVWESGCVVIVMLTPLAENGVRQCYHYWPDEGSNLYHIYEVNLVSEHIWCED |
| | | FLVRSFYLKNLQTNETRTVTQFHFLSWYDRGVPSSSRSLLDFRRKVNKCYRG |
| | | RSCPIIVHCSDGAGRSGTYVLIDMVLNKMAKGAKEIDIAATLEHLRDQRPGM |
| | | VQTKEQFEFALTAVAEEVNAILKALPQ |
| glutamic acid | 7 | MASSTPSSSATSSNAGADPNTTNLRPTTYDTWCGVAHGCTRKLGLKICGFLQ |
| decarboxylase 67- | | RTNSLEEKSRLVSAFKERQSSKNLLSCENSDRDARFRRTETDFSNLFARDLL |
| kDa isoform | | PAKNGEEQTVQFLLEVVDILLNYVRKTFDRSTKVLDFHHPHQLLEGMEGENL |
| (GAD67) | | ELSDHPESLEQILVDCRDTLKYGVRTGHPRFFNQLSTGLDIIGLAGEWLTST |
| | | ANTNMFTYEIAPVFVLMEQITLKKMREIVGWSSKDGDGIFSPGGAISNMYSI |
| | | MAARYKYFPEVKTKGMAAVPKLVLFTSEQSHYSIKKAGAALGFGTDNVILIK |
| | | CNERGKIIPADFEAKILEAKQKGYVPFYVNATAGTTVYGAFDPIQEIADICE |
| | | KYNLWLHVDAAWGGGLLMSRKHRHKLNGIERANSVTWNPHKMMGVLLQCSAI |
| | | LVKEKGILQGCNQMCAGYLFQPDKQYDVSYDTGDKAIQCGRHVDIFKFWLMW |
| | | KAKGTVGFENQINKCLELAEYLYAKIKNREEFEMVENGEPEHTNVCFWYIPQ |
| | | SLRGVPDSPQRREKLHKVAPKIKALMMESGTTMVGYQPQGDKANFFRMVISN |
| | | PAATQSDIDFLIEEIERLGQDL |
| Islet cell antigen-69 | 8 | MSGHKCSYPWDLQDRYAQDKSVVNKMQQKYWETKQAFIKATGKKEDEHVVAS |
| (ICA69) | | DADLDAKLELFHSIQRTCLDLSKAIVLYQKRICFLSQEENELGKFLRSQGFQ |
| | | DKTRAGKMMQATGKALCFSSQQRLALRNPLCRFHQEVETFRHRAISDTWLTV |
| | | NRMEQCRTEYRGALLWMKDVSQELDPDLYKQMEKFRKVQTQVRLAKKNEDKL |
| | | KMDVCQKVDLLGASRCNLLSHMLATYQTTLLHFWEKTSHTMAAIHESFKGYQ |
| | | PYEFTTLKSLQDPMKKLVEKEEKKKINQQESTDAAVQEPSQLISLEEENQRK |
| | | ESSSFKTEDGKSILSALDKGSTHTACSGPIDELLDMKSEEGACLGPVAGTPE |
| | | PEGADKDDLLLLSEIFNASSLEEGEFSKEWAAVFGDGQVKEPVPTMALGEPD |
| | | PKAQTGSGFLPSQLLDQNMKDLQASLQEPAKAASDLTAWFSLFADLDPLSNP |
| | | DAVGKTDKEHELLNA |
| Chromogranin A | 9 | MRSAAVLALLLCAGQVTALPVNSPMNKGDTEVMKCIVEVISDTLSKPSPMPV |
| | | SQECFETLRGDERILSILRHQNLLKELQDLALQGAKERAHQQKKHSGFEDEL |
| | | SEVLENQSSQAELKEAVEEPSSKDVMEKREDSKEAEKSGEATDGARPQALPE |
| | | PMQESKAEGNNQAPGEEEEEEEEATNTHPPASLPSQKYPGPQAEGDSEGLSQ |
| | | GLVDREKGLSAEPGWQAKREEEEEEEEEAEAGEEAVPEEEGPTVVLNPHPSL |
| | | GYKEIRKGESRSEALAVDGAGKPGAEEAQDPEGKGEQEHSQQKEEEEEMAVV |
| | | PQGLFRGGKSGELEQEEERLSKEWEDSKRWSKMDQLAKELTAEKRLEGQEEE |
| | | EDNRDSSMKLSFRARAYGFRGPGPQLRRGWRPSSREDSLEAGLPLQVRGYPE |
| | | EKKEEEGSANRRPEDQELESLSAIEAELEKVAHQLQALRRG |
| Chromogranin A | 10 | MRSAAVLALLLCAGQVTALPVNSPMNKGDTEVMKCIVEVISDTLSKPSPMPV |
| isoform | | SQECFETLRGDERILSILRHQNLLKELQDLALQGAKERAHQQKKHSGFEDEL |
| | | SEVLENQSSQAELKGRSEALAVDGAGKPGAEEAQDPEGKGEQEHSQQKEEEE |
| | | EMAVVPQGLFRGGKSGELEQEEERLSKEWEDSKRWSKMDQLAKELTAEKRLE |
| | | GQEEEEDNRDSSMKLSFRARAYGFRGPGPQLRRGWRPSSREDSLEAGLPLQV |
| | | RGYPEEKKEEEGSANRRPEDQELESLSAIEAELEKVAHQLQALRRG |
| Peripherin | 11 | MSHHPSGLRAGFSSTSYRRTFGPPPSLSPGAFSYSSSSRFSSSRLLGSASPS |
| | | SSVRLGSFRSPRAGAGALLRLPSERLDFSMAEALNQEFLATRSNEKQELQEL |
| | | NDRFANFIEKVRFLEQQNAALRGELSQARGQEPARADQLCQQELRELRRELE |
| | | LLGRERDRVQVERDGLAEDLAALKQRLEEETRKREDAEHNLVLFRKDVDDAT |
| | | LSRLELERKIESLMDEIEFLKKLHEEELRDLQVSVESQQVQQVEVEATVKPE |
| | | LTAALRDIRAQYESIAAKNLQEAEEWYKSKYADLSDAANRNHEALRQAKQEM |
| | | NESRRQIQSLTCEVDGLRGTNEALLRQLRELEEQFALEAGGYQAGAARLEEE |
| | | LRQLKEEMARHLREYQELLNVKMALDIEIATYRKLLEGEESRISVPVHSFAS |
| | | LNIKTTVPEVEPPQDSHSRKTVLIKTIETRNGEVVTESQKEQRSELDKSSAH |
| | | SY |
| Peripherin-2 | 12 | MALLKVKFDQKKRVKLAQGLWLMNWFSVLAGIIIFSLGLFLKIELRKRSDVM |
| | | NNSESHFVPNSLIGMGVLSCVFNSLAGKICYDALDPAKYARWKPWLKPYLAI |
| | | CVLFNIILFLVALCCFLLRGSLENTLGQGLKNGMKYYRDTDTPGRCFMKKTI |
| | | DMLQIEFKCCGNNGFRDWFEIQWISNRYLDFSSKEVKDRIKSNVDGRYLVDG |
| | | VPFSCCNPSSPRPCIQYQITNNSAHYSYDHQTEELNLWVRGCRAALLSYYSS |
| | | LMNSMGVVTLLIWLFEVTITIGLRYLQTSLDGVSNPEESESESEGWLLEKSV |
| | | PETWKAFLESVKKLGKGNQVEAEGAGAGQAPEAG |
| Islet amyloid peptide | 13 | MGILKLQVFLIVLSVALNHLKATPIESHQVEKRKCNTATCATQRLANFLVHS |
| (ppIAPP) | | SNNFGAILSSTNVGSNTYGKRNAVEVLKREPLNYLPL |
| carboxypeptidase | 14 | MAGRGGSALLALCGALAACGWLLGAEAQEPGAPAAGMRRRRRLQQEDGISFE |
| H/E | | YHRYPELREALVSVWLQCTAISRIYTVGRSFEGRELLVIELSDNPGVHEPGE |
| | | PEFKYIGNMHGNEAVGRELLIFLAQYLCNEYQKGNETIVNLIHSTRIHIMPS |
| | | LNPDGFEKAASQPGELKDWFVGRSNAQGIDLNRNFPDLDRIVYVNEKEGGPN |
| | | NHLLKNMKKIVDQNTKLAPETKAVIHWIMDIPFVLSANLHGGDLVANYPYDE |
| | | TRSGSAHEYSSSPDDAIFQSLARAYSSFNPAMSDPNRPPCRKNDDDSSFVDG |
| | | TTNGGAWYSVPGGMQDFNYLSSNCFEITVELSCEKFPPEETLKTYWEDNKNS |
| | | LISYLEQIHRGVKGFVRDLQGNPIANATISVEGIDHDVTSAKDGDYWRLLIP |
| | | GNYKLTASAPGYLAITKKVAVPYSPAAGVDFELESFSERKEEEKEELMEWWK |
| | | MMSETLNF |
| Heat shock protein | 15 | MLRLPTVFRQMRPVSRVLAPHLTRAYAKDVKFGADARALMLQGVDLLADAVA |
| 60 (Hsp60) | | VTMGPKGRTVIIEQSWGSPKVTKDGVTVAKSIDLKDKYKNIGAKLVQDVANN |
| | | TNEEAGDGTTTATVLARSIAKEGFEKISKGANPVEIRRGVMLAVDAVIAELK |
| | | KQSKPVTTPEEIAQVATISANGDKEIGNIISDAMKKVGRKGVITVKDGKTLN |
| | | DELEIIEGMKFDRGYISPYFINTSKGQKCEFQDAYVLLSEKKISSIQSIVPA |
| | | LEIANAHRKPLVIIAEDVDGEALSTLVLNRLKVGLQVVAVKAPGFGDNRKNQ |
| | | LKDMAIATGGAVFGEEGLTLNLEDVQPHDLGKVGEVIVTKDDAMLLKGKGDK |
| | | AQIEKRIQEIIEQLDVTTSEYEKEKLNERLAKLSDGVAVLKVGGTSDVEVNE |
| | | KKDRVTDALNATRAAVEEGIVLGGGCALLRCIPALDSLTPANEDQKIGIEII |
| | | KRTLKIPAMTIAKNAGVEGSLIVEKIMQSSSEVGYDAMAGDFVNMVEKGIID |
| | | PTKVVRTALLDAAGVASLLTTAEVVVTEIPKEEKDPGMGAMGGMGGGMGGGM |
| | | F |
| Zinc transporter 8 | 16 | MEFLERTYLVNDKAAKMYAFTLESVELQQKPVNKDQCPRERPEELESGGMYH |
| (ZnT8), variant | | CHSGSKPTEKGANEYAYAKWKLCSASAICFIFMIAEVVGGHIAGSLAVVTDA |
| | | AHLLIDLTSFLLSLFSLWLSSKPPSKRLTFGWHRAEILGALLSILCIWVVTG |
| | | VLVYLACERLLYPDYQIQATVMIIVSSCAVAANIVLTVVLHQRCLGHNHKEV |
| | | QANASVRAAFVHALGDLFQSISVLISALIIYFKPEYKIADPICTFIFSILVL |
| | | ASTITILKDFSILLMEGVPKSLNYSGVKELILAVDGVLSVHSLHIWSLTMNQ |
| | | VILSAHVATAASRDSQVVRREIAKALSKSFTMHSLTIQMESPVDQDPDCLFC |
| | | EDPC |
| Heat shock protein | 17 | AKAAAIGIDLGTTYSCVGVFQHGKVEIIANDQGNRTTPSYVAFTDTERLIGD |
| 70 A1 (HSPA1A) | | AAKNQVALNPQNTVFDAKRLIGRKFGDPVVQSDMKHWPFQVINDGDKPKVQV |
| | | SYKGETKAFYPEEISSMVLTKMKEIAEAYLGYPVTNAVITVPAYENDSQRQA |
| | | TKDAGVIAGLNVLRIINEPTAAAIAYGLDRTGKGERNVLIFDLGGGTFDVSI |
| | | LTIDDGIFEVKATAGDTHLGGEDFDNRLVNHFVEEFKRKHKKDISQNKRAVR |
| | | RLRTACERAKRTLSSSTQASLEIDSLFEGIDFYTSITRARFEELCSDLFRST |
| | | LEPVEKALRDAKLDKAQIHDLVLVGGSTRIPKVQKLLQDFFNGRDLNKSINP |
| | | DEAVAYGAAVQAAILMGDKSENVQDLLLLDVAPLSLGLETAGGVMTALIKRN |
| | | STIPTKQTQIFTTYSDNQPGVLIQVYEGERAMTKDNNLLGRFELSGIPPAPR |
| | | GVPQIEVTFDIDANGILNVTATDKSTGKANKITITNDKGRLSKEEIERMVQE |
| | | AEKYKAEDEVQRERVSAKNALESYAFNMKSAVEDEGLKGKISEADKKKVLDK |
| | | CQEVISWLDANTLAEKDEFEHKRKELEQVCNPIISGLYQGAGGPGPGGFGAQ |
| | | GPKGGSGSGPTIEEVD |

In various embodiments, TIMP-T1D is administered in a single dose or in multiple doses. In various embodiments, TIMP-T1D is administered in two doses one-week apart. In various embodiments, TIMP-T1D is administered once weekly, once every two weeks, once every three weeks, once every 4 weeks, once every two months, once every three months, once every 6 months, or once per year.

In various embodiments, TIMP-T1D comprises poly (lactic co-glycolic acid) (PLGA) particles encapsulating one or more T1D antigens and a suitable buffering agent or excipient. In various embodiments, TIMP-T1D consists of poly (lactic co-glycolic acid) (PLGA) particles encapsulating one or more T1D antigens and a suitable buffering agent or excipient. In various embodiments, TIMP-T1D particles are surface functionalized. In various embodiments, TIMP-T1D particles are surface functionalized by carboxylation. In various embodiments, TIMP-T1D particles have a negative zeta potential. In various embodiments, the negative zeta potential of TIMP-T1D particles is between about −100 mV to about 0 mV. In various embodiments, the zeta potential of the particles is from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV including all ranges and values lying between that range.

In various embodiments, the size, or diameter, of TIMP-T1D particles is between 0.05 μm to about 10 μm. In various embodiments, the diameter of TIMP-T1D particles is between 0.1 μm and about 10 μm. In various embodiments, the diameter of TIMP-T1D particles is between 0.1 μm and about 5 μm. In various embodiments, the diameter of TIMP-T1D particles is between 0.1 μm and about 3 μm. In various embodiments, the diameter of TIMP-T1D particles is between 0.3 μm and about 5 μm. In various embodiments, the diameter of TIMP-T1D particles is about 0.3 μm to about 3 μm. In various embodiments, the diameter of TIMP-T1D particles is between about 0.3 μm to about 1 μm. In various embodiments, the diameter of TIMP-T1D particles is between about 0.4 μm to about 1 μm. In various embodiments, the TIMP-T1D particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-T1D particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 400 nm to 800 nm. In various embodiments, the particles have a size between about 400 to about 800 nm and a zeta potential between −30 mV and −80 mV. In various embodiments, the polydispersity index (PDI) or heterogeneity index for particle size is between 0.01 and 1.0 (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 including all values within the range).

In various embodiments, the particles have a homogenous size distribution. In various embodiments, the particles have a homogenous size distribution wherein at least 90% of the particles have a diameter of between 0.05 μm and about 10 μm, between 0.1 μm and about 10 μm, 0.1 μm and about 5 μm, 0.1 μm and about 3 μm, 0.3 μm and about 5 μm, 0.3 μm to about 3 μm. In various embodiments, the particles have a homogenous size distribution wherein at least 90% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-T1D particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 50% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm. In various embodiments, the particles have a homogenous size distribution wherein at least 50% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-T1D particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 10% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm. In various embodiments, the particles have a homogenous size distribution wherein at least 10% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-T1D particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm.

In various embodiments, TIMP-T1D particles useful in the methods or compositions encapsulate T1D-associated antigens comprising PPI, ZnT8, GAD65, and IGRP, have a negative zeta potential between −30 mV to −80 mV and have a diameter between 400 nm and 800 nm.

In various embodiments, TIMP-T1D encapsulates T1D-associated antigens comprising SEQ ID NO: 1, SEQ ID NO: 2, SEQ ID NO: 3, and SEQ ID NO: 4, have a negative zeta potential between −30 mV to −80 mV and have a diameter between 400 nm and 800 nm. In various embodiments, TIMP-T1D encapsulates T1D-associated antigens comprising SEQ ID NO: 1, SEQ ID NO: 2, SEQ ID NO: 3, and SEQ ID NO: 4, have a negative zeta potential between −30 mV to −80 mV and have a diameter between 400 nm and 800 nm. In various embodiments, TIMP-T1D encapsulates T1D-associated antigens comprising SEQ ID NO: 1, SEQ ID NO: 2, SEQ ID NO: 3, and SEQ ID NO: 16, have a negative zeta potential between −30 mV to −80 mV and have a diameter between 400 nm and 800 nm.

In various embodiments, TIMP-T1D is administered intravenously, subcutaneously, intramuscularly, intraperitoneally, intranasally, intra-lymphatically, or orally. In various embodiments, TIMP-T1D is administered intravenously, subcutaneously, intramuscularly, intraperitoneally, intranasally, inhaled, intra-lymphatically, or orally.

In various embodiments, TIMP-T1D is administered at a concentration between 0.005 mg/mL and 50 mg/mL inclusive of all values and ranges between these values. In various embodiments, TIMP-T1D is administered at a concentration of about 0.05 mg/mL, about 0.1 mg/mL, about 0.5 mg/mL, about 1 mg/mL, about 2 mg/mL, about 3 mg/mL, about 3.25 mg/ml, about 3.5 mg/ml, about 4 mg/mL, about 5 mg/mL, about 6 mg/mL, about 7 mg/mL, about 8 mg/mL, about 9 mg/mL, about 10 mg/mL, about 11 mg/mL, about 12.5 mg/mL, about 15 mg/mL, about 17.5 mg/mL, about 20 mg/mL, about 25 mg/mL, about 30 mg/mL, about 40 mg/mL, or about 50 mg/mL. In various embodiments, TIMP-T1D is administered via intravenous infusion lasting about 1, 2, 3, 4, 5, 6, 7, or 8 hours.

In various embodiments, administering TIMP-T1D to a subject in need thereof, relieves one or more symptoms of T1D. In various embodiments, the symptoms of T1D are selected from the group consisting of increased or frequent urination, increased thirst, increased hunger, extreme fatigue, blurry vision, loss of muscle, cuts or bruises that are slow to heal, coronary artery disease with chest pain (angina), heart attack, stroke, atherosclerosis, high blood pressure, nerve damage (neuropathy), kidney damage (nephropathy), diabetic retinopathy, diabetic ketoacidosis, foot damage, increased bacterial and viral infection in skin and mouth, gum disease, pregnancy complications like miscarriage, stillbirth and birth defects, exogenous insulin use, hypoglycemic events, elevated HbA1c, deteriorating β-cell function, declining C-peptide levels, decreased time in glycemic target range of 70-110 mg/dl (3.9 to 6.1 mM/L) glucose i.e. hyperglycemia, decreased time in glycemic target range of 70-180 mg/dL (3.9 to 10.0 mM/L) glucose, microvascular complications, and weight loss.

In various embodiments, administering TIMP-T1D to a subject in need thereof decreases the duration and severity of an autoimmune or inflammatory immune response to one or more T1D antigens. In various embodiments, the inflammatory immune response is a T cell response, B cell response, Th1 response, myeloid cell response, and/or an antibody response. In various embodiments, the efficacy of TIMP-T1D at relieving one or more symptoms of T1D and/or reducing the duration and severity of an inflammatory immune response to one or more T1D antigens is determined from the assay of one or more biological samples from the subject. In various embodiments, the biological samples are selected from the group consisting whole-blood, peripheral blood, peripheral blood mononuclear cells (PBMCs), serum, plasma, urine, cerebrospinal fluid (CSF), stool, a tissue biopsy, and/or a bone-marrow biopsy.

In various embodiments, administering TIMP-T1D in a subject decreases levels of antigen-specific T cells relative to the subject's baseline measurement and/or relative to a placebo control. In various embodiments, administering TIMP-T1D in a subject decreases level of antigen-specific CD4+ T cells relative to the subject's baseline measurement and/or relative to a placebo control. In various embodiments, administering TIMP-T1D in a subject decreases levels of antigen-specific CD8+ T cells relative to the subject's baseline measurement and/or relative to a placebo control. In various embodiments, administering TIMP-T1D in a subject stabilizes levels of antigen-specific T cells relative to the subject's baseline measurement and/or relative to a placebo control. In various embodiments, administering TIMP-T1D in a subject stabilizes levels of antigen-specific CD4+ and/or CD8+ T cells relative to the subject's baseline measurement and/or relative to a placebo control.

In various embodiments, administering TIMP-T1D in a subject decreases levels of activated antigen-specific T cells relative to the subject's baseline measurement and/or relative to a placebo control. In various embodiments, administering TIMP-T1D in a subject decreases levels of activated antigen-specific CD4+ and/or CD8+ T cells relative to the subject's baseline measurement and/or relative to a placebo control. In various embodiments, administering TIMP-T1D in a subject stabilizes levels of activated antigen-specific T cells relative to the subject's baseline measurement and/or relative to a placebo control. In various embodiments, administering TIMP-T1D in a subject stabilizes levels of activated antigen-specific CD4+ and/or CD8+ T cells relative to the subject's baseline measurement and/or relative to a placebo control.

In various embodiments, administering TIMP-T1D in a subject increases levels of antigen-specific regulatory T (Treg) cells relative to the subject's baseline measurement and/or relative to a placebo control. In various embodiments, administering TIMP-T1D in a subject increases levels of antigen-specific CD4+ Tregs relative to the subject's baseline measurement and/or relative to a placebo control. In various embodiments, administering TIMP-T1D in a subject increases levels of antigen-specific CD8+ Tregs relative to the subject's baseline measurement and/or relative to a placebo control.

In various embodiments, administering TIMP-T1D in a subject stabilizes C-peptide levels relative to the subject's baseline measurement and/or relative to a placebo control. In various embodiments, administering TIMP-T1D in a subject increases C-peptide levels relative to the subject's baseline measurement and/or relative to a placebo control.

In various embodiments, administering TIMP-T1D in a subject stabilizes area under the curve (AUC) of stimulated C-peptide response in a mixed meal tolerance test (MMTT) relative to the subject's baseline measurement and/or relative to a placebo control. In various embodiments, administering TIMP-T1D in a subject delays decline in under the curve (AUC) of stimulated C-peptide response in a mixed meal tolerance test (MMTT) relative to the subject's baseline measurement and/or relative to a placebo control. In various embodiments, administering TIMP-T1D in a subject increases area under the curve (AUC) of stimulated C-peptide response in a mixed meal tolerance test (MMTT) relative to the subject's baseline measurement and/or relative to a placebo control. In various embodiments, administering TIMP-T1D in a subject stabilizes or increases C-peptide levels in a glucagon stimulation test. In various embodiments, administering TIMP-T1D in a subject stabilizes or increases change in fasting C-peptide levels in a glucagon stimulation test.

In various embodiments, administering TIMP-T1D in a subject lowers HbA1c levels relative to the subject's baseline measurement and/or relative to a placebo control. In various embodiments, administering TIMP-T1D in a subject stabilizes HbA1c levels relative to the subject's baseline measurement and/or relative to a placebo control. In various embodiments, administering TIMP-T1D in a subject stabilizes or lowers insulin-dose adjusted A1c (IDAA1c). In various embodiments, administering TIMP-T1D in a subject stabilizes or lowers insulin-dose adjusted A1c (IDAA1c) to ≤9. In various embodiments, administering TIMP-T1D in a subject alters insulin sensitivity score (ISS).

In various embodiments, administering TIMP-T1D in a subject lowers, or stabilizes glycemic variability/fluctuations. In various embodiments, administering TIMP-T1D in a subject increases subject's time in glycemic target range of 70-110 mg/dL (3.9 to 6.1 mmol/L) glucose relative to the subject's baseline measurement and/or relative to a placebo control. In various embodiments, administering TIMP-T1D in a subject stabilizes subject's time in glycemic target range of 70-110 mg/dL (3.9 to 6.1 mmol/L) glucose relative to the subject's baseline measurement and/or relative to a placebo control. In various embodiments, glycemic range is measured by continuous glucose monitoring. In various embodiments, administering TIMP-T1D in a subject increases subject's time in glycemic target range of 70-180 mg/dL (3.9 to 10.0 mM/L) glucose relative to the subject's baseline measurement and/or relative to a placebo control. In various embodiments, administering TIMP-T1D in a subject stabilizes subject's time in glycemic target range of 70-180 mg/dL (3.9 to 10.0 mM/L) glucose relative to the subject's baseline measurement and/or relative to a placebo control. In various embodiments, glycemic range is measured by continuous glucose monitoring.

In various embodiments, administering TIMP-T1D in a subject decreases exogenous insulin use relative to the subject's baseline measurement and/or relative to a placebo control. In various embodiments, administering TIMP-T1D in a subject stabilizes exogenous insulin use relative to the subject's baseline measurement and/or relative to a placebo control.

In various embodiments, administering TIMP-T1D in a subject decreases hypoglycemic events relative to the subject's baseline measurement and/or relative to a placebo control. In various embodiments, administering TIMP-T1D in a subject stabilizes hypoglycemic events relative to the subject's baseline measurement and/or relative to a placebo control.

In various embodiments, administering TIMP-T1D in a subject decreases blood sugar levels to the subject's baseline measurement and/or relative to a placebo control. In various embodiments, administering TIMP-T1D in a subject stabilizes blood glucose levels relative to the subject's baseline measurement and/or relative to a placebo control.

In various embodiments, the disclosure provides a method of treating T1D or T1D related symptoms, in a subject in need thereof comprising administering to the subject a composition comprising TIMP-T1D alone or in combination with one or more therapeutics useful to treat T1D. In various embodiments, the combination therapeutics useful to treat T1D are selected from the group consisting of insulin, insulin pumps, high blood pressure medication, aspirin, cholesterol-lowering drugs, artificial pancreas, anti-IL-21 antibody, B-lymphocyte depletion (e.g. rituximab), anti-CD3 antibody (e.g. teplizumab), adoptive transfer of Tregs (e.g. autologous Tregs, umbilical cord derived Tregs, polyclonal Tregs), antithymocyte globulin, sodium-glucose cotransporter-2 (SGLT2) inhibitors (e.g. dapagliflozin, sotagliflozin), stem cell mobilization (e.g. Plerixafor), β-cell transplant, islet transplant, stem cell transplant, incretins therapy (e.g. liraglutide). In various embodiments, the insulin is selected from the group consisting of insulin glargine, rapid acting insulin (e.g. insulin aspart, insulin lispro, insulin glulisine), short acting insulin (ACTRAPID®, HUMULIN S®, INSUMAN RAPID®), long acting insulin (LEVEMIR®, ABASAGLAR®, LANTUS®, TOUJEO®, TRESIBA®) or intermediate acting (INSULATARD®, INSUMAN BASAL®).

In various embodiments, the combination therapeutic induces Tregs. In various embodiments, the therapeutic increases the frequency and/or the number Tregs. In various embodiments, the therapeutic is IL-2 therapy to induce Tregs. In various embodiments, the IL-2 therapy is low dose IL-2, IL-2 muteins engineered to expand Tregs, IL-2 variants engineered to expand Tregs, IL-2 molecules engineered to be selective for the high-affinity IL-2 receptor, PEGylated IL-2, IL-2 complexes, or IL-2/CD25 fusion proteins,

In various embodiments, the one or more therapeutics are administered prior to, concomitantly with or subsequent to the administration of TIMP-T1D. In various embodiments, the therapeutics are administered 0.5 to 23 hours prior to administration of TIMP-T1D. In various embodiments, the therapeutics are administered 1, 2, 3, 4, 5, 6, or 7 days prior to administration of TIMP-T1D. In various embodiments, the therapeutics are administered 1, 2, 3, or 4 weeks prior to administration of TIMP-T1D. In various embodiments, the therapeutics are administered 1, 2, 3, 4, 5, 6, 7, 8, 9, 10, 11, or 12 months prior to administration of TIMP-T1D. In various embodiments, the therapeutics are administered 1, 2, 3, 4, 5, 6, 7, 8, 9, 10, 11, or 12 years prior to administration of TIMP-T1D. In various embodiments, the therapeutics are administered 0.5 to 24 hours subsequent to administration of TIMP-T1D. In various embodiments, the therapeutics are administered 1, 2, 3, 4, 5, 6, or 7 days subsequent to administration of TIMP-T1D. In various embodiments, the therapeutics are administered 1, 2, 3, or 4 weeks subsequent to administration of TIMP-T1D. In various embodiments, the therapeutics are administered 1, 2, 3, 4, 5, 6, 7, 8, 9, 10, 11, or 12 months subsequent to administration of TIMP-T1D. In various embodiments, the therapeutics are administered 1, 2, 3, 4, 5, 6, 7, 8, 9, 10, 11, or 12 years subsequent to administration of TIMP-T1D.

Also contemplated is a composition comprising TIMP-T1D as described herein for use in treating type 1 diabetes. In various embodiments, the disclosure provides for use of a composition comprising TIMP-T1D as described herein in the preparation of a medicament for treating type 1 diabetes.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1. Schedule of events for the clinical trial evaluating safety and efficacy of CNP-103.

FIG. 2A-2B. Pre-clinical dosing of CNP-T1D in NOD mice with booster doses. Diabetic NOD mice were treated with two loading doses of CNP-T1D followed by a booster dose every 4 weeks. Treatment of mice with CNP-T1D significantly inhibited the development of T1D in dysglycemic NOD mice with further inhibition with booster doses (FIG. 2A). Treatment of mice with either 2 or 5 doses of 2.5 mg CNP-T1D significantly improved protection from T1D in dysglycemic NOD mice compared with control saline treated NOD mice. (FIG. 2B) (*** p<0.0005).

FIG. 3. Reversal of dysglycemia (blood glucose ≥150 mg/dL and <250 mg/dL) was more pronounced in the NOD mice that received the booster dosing schedule with 5 doses compared to mice that received just the 2 loading doses. Comparison between CNP-T1D at 2.5 mg dose, 2 doses versus 5 doses, was significant. (*p=0.0183).

FIG. 4: Schedule of events for the clinical trial evaluating safety and efficacy of three doses of CNP-103.

### DETAILED DESCRIPTION

There is a need for therapeutics for addressing the immune imbalance in T1D leading to improved disease symptoms and improved outcomes without the risk of toxic side-effects. The present disclosure provides methodology for monitoring the induction of and maintenance of immunologic tolerance in a subject having T1D after receiving immunotherapy.

The headings herein are for the convenience of the reader and not intended to be limiting. Additional aspects, embodiments, and variations of the invention will be apparent from the Detailed Description and/or Drawing and/or claims.

### DEFINITIONS

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term “particle” may be used to encompass beads and spheres.

“Negatively charged particle” as used herein refers to particles which have been modified to possess a net surface charge that is less than zero.

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups.

As used herein, the term “Th cell” or “helper T cell” refers to CD4+ cells. CD4+ T cells assist other white blood cells with immunologic processes, including maturation of B cells into plasma cells and memory B cells, and activation of cytotoxic T cells and macrophages. T cells become activated when they are presented with peptide antigens by MHC class II molecules, which are expressed on the surface of antigen-presenting cells (APCs).

As used herein, the term “Th1 cell” refers to a subset of Th cells which produce proinflammatory mediators. Th1 cells secrete cytokines to facilitate immune response and play a role in host defense against pathogens in part by mediating the recruitment of neutrophils and macrophages to infected tissues. Th1 cells secrete cytokines including IFN-gamma, IL2, IL-10, and TNF alpha/beta to coordinate defense against intracellular pathogens such as viruses and some bacteria.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally-occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via host cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure and/or amino acid sequence that is identical or is similar to the epitope used to generate the antibody, yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure and/or the amino acid sequence of the epitope and mimotope.

The term “symptom” is used herein to mean any physical or observable manifestation of a disorder, whether it is generally characteristic of that disorder or not. The term “symptoms” can mean all such manifestations or any subset thereof.

The term “therapeutically effective amount” is used herein to indicate the amount of antigen-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, cither temporarily or permanently, either partially or completely, one or more clinical symptom, manifestation or progression of an event, disease or condition. Such treating need not be absolute to be useful. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

Hypoglycemia or “typical” hypoglycemia can be defined as blood sugar levels ≥54 mg/dL (3.0 mmol/L) but <70 mg/dL (3.9 mmol/L). Severe hypoglycemia of clinically significant hypoglycemic events are defined as blood sugar levels <54 mg/dL.

Hyperglycemia can be defined as blood sugar levels >180 mg/dL but ≤250 mg/dL (>10 mmol/L but ≤13.9 mmol/L). Severe hyperglycemia is >250 mg/dL. In classic symptoms, a plasma glucose level of ≥200 mg/dL (11.1 mmol/L). Hyperglycemia can also be defined as fasting plasma glucose levels >110 mg/dL.

Insulin-dose-adjusted HbA1c (IDAA1c) may be calculated as HbA1c (%)+4× insulin dose (units per kilogram per 24 h).

Insulin sensitivity score maybe calculated as loge IS=4:64725-0:02032δwaist, cmp-0:09779 (HbA1c, %-0:00235 (TG, MG/DL), wherein TG is triglycerides.

### Particles

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a negative charge of between 0 to about −100 mV. In various embodiments, the particles are 400-800 nanometers in diameter and have a charge of between about −25 mV and −70 mV. In various embodiments, the particles are 400-800 nanometers in diameter and have a charge of between about −30 mV and −80 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PGA particles, PLA particles, PLGA particles, PLURIONICS stabilized polypropylene sulfide particles, polypropylene sulfone particles, poly (ethylene glycol)-block-poly(propylene sulfide) copolymer particles and diamond particles. In some embodiments, the liposome encapsulates polynucleotides encoding T1D associated antigens. In various embodiments, the polynucleotides consist of DNA, RNA, mRNA, or circular RNA.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly (ethylene glycol)) may decrease the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22:1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0, or about 20:80 to 100:0. Suitable co-polymer ratio of present tolerizing immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In certain embodiments, the particle is a PLURONICS stabilized polypropylene sulfide particle, a polyglycolic acid particle (PGA), a polylactic acid particle (PLA), or a poly(lactic-co-glycolic acid) particle, or a carboxlated polyglycolic acid particle (PGA), carboxylated polylactic acid particle (PLA), or carboxylated poly(lactic-co-glycolic acid) particle. In certain embodiments, the particle has a copolymer ratio of polylactic acid/polyglycolic acid 80:20: polylactic acid/polyglycolic acid 90:10: polylactic acid/polyglycolic acid 50:50. In various embodiments, the particle is a poly(lactic-co-glycolic acid) particle and has a copolymer ratio of about 50:50 polylactic acid: polyglycolic acid.

It is contemplated that the particle may further comprise a surfactant. The surfactant can be anionic, cationic, zwitterionic or nonionic. Surfactants in the poloxamer and poloaxamines family are commonly used in particle synthesis. Surfactants that may be used, include, but are not limited to PEG, Tween-80, gelatin, dextran, pluronic L-63, poly vinyl alcohol (PVA), poly acrylic acid (PAA), methylcellulose, lecithin, didodecyldimethylammonium bromide (DMAB) and poly(ethylene-alt-maleic acid) (PEMA). Additionally, biodegradable and biocompatible surfactants including, but not limited to, vitamin E TPGS (D-a-tocopheryl polyethylene glycol 1000 succinate), poly amino acids (e.g polymers of lysine, arginine, aspartic acid, glutamic acid, serine, threonine, tyrosine and cysteine, or their enantiomers), and sulfate polymers. In certain embodiments, two surfactants are used. For example, if the particle is produced by a double emulsion method, the two surfactants can include a hydrophobic surfactant for the first emulsion, and a hydrophobic surfactant for the second emulsion. In certain embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to decrease the nanoparticle size. One strategy to accomplish this is to apply a second strong shear rate. The leakage effect can be decreased by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference.

### Antigens

An antigen refers to a discreet portion of a molecule, such as a polypeptide or peptide sequence, a 3-dimensional structural formation of a polypeptide or peptide, a polysaccharide or polynucleotide that can be recognized by a host immune cells. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity or re-programming of T cells to T cell receptor-mediated stimulation. Such reprogramming is generally antigen-specific and persists after exposure to the antigenic peptide has ceased. This reprogramming leads to induction of regulatory T cells, Tr1 cells and T cell anergy. For example, insensitivity in T cells is characterized by lack of effector cytokine production, lack of proliferation, or lack of activation. Reprogramming occurs when T cells are exposed to antigen and receive a first signal (a T cell receptor or CD3 mediated signal) in the absence of a second signal (a costimulatory signal), or presence of an inhibitory signal (negative costimulation or regulatory cytokines). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more T1D antigens. The one or more T1D antigens may be whole proteins, polypeptides, or peptides comprising T1D antigenic epitopes.

In certain embodiments, one, two, three, or a higher number of antigens or antigenic peptides are used in the TIMPs. In certain embodiments, the one or more T1D antigens is encapsulated in the TIMP by covalent linkage to the interior surface of the particle (See e.g., US Patent Publication US20190282707, herein incorporated by reference). In certain embodiments, it is contemplated that sequences of two or more T1D antigens are linked in a fusion protein and encapsulated within a TIMP described herein. Methods for making TIMP with linked epitopes are described in US Patent Publication US20190365656, herein incorporated by reference.

### Methods of Use

Type 1 diabetes (T1D, also referred to as type 1 diabetes mellitus) is a T-cell driven autoimmune disease directed towards β-cells of the pancreas wherein a combination of T cell and B cell activation results in islet β-cell loss. The prevalence of T1D in the United States is estimated at approximately 1.6 million people. There is a significant unmet medical need with this disease and an opportunity where TIMP-T1D will positively impact subjects suffering from this disease.

The most common cause of death in patients with T1D is due to complications of the condition. Acute, or sudden, complications include low blood sugar (hypoglycemia) and ketoacidosis. Chronic complications include heart disease, stroke, neuropathy, nephropathy, need for amputation of toes, feet or legs, and diabetic retinopathy. Current standard of care treatment for T1D is insulin therapy that can result in hypo- and hyperglycemia and diabetic ketoacidosis.

Treatments currently in clinical trials for T1D fall into these categories: anti-IL-21 antibody, islet and stem cell transplants, peptide therapies, and immune suppressants such as rituximab and abatacept. However, these classes of therapies do not address the underlying cause of pathology that is driven by antigen specific autoimmune attack on the pancreas.

Provided herein is a method of treating T1D in a subject comprising administering to the subject TIMP-T1D, wherein TIMP-T1D is administered at a dose level determined based on the subject's weight. It is also contemplated that TIMP-T1D may be administered at a fixed dosage irrespective of the subject's weight. In various embodiments, contemplated is a method of treating T1D in a subject comprising administering to the subject TIMP-T1D, wherein TIMP-T1D is administered at a dose of 0.01 to 12 mg/kg inclusive of all values and ranges between these values based on the subject's weight or at a fixed dose between 1 mg and 800 mg inclusive of all values and ranges between these values. Also provided herein is a method of reducing an inflammatory immune response to T1D antigens in a subject suffering from T1D comprising administering to the subject TIMP-T1D, wherein TIMP-T1D is administered at a dose of 0.01 to 12 mg/kg inclusive of all values and ranges between these values based on the subject's weight or at a fixed dose between 1 mg and 800 mg inclusive of all values and ranges between these values.

Also contemplated, the TIMP-T1D is administered at a dose from about 0.01 to about 12 mg/kg, from about 0.05 to about 10 mg/kg, from about 0.01 to about 5 mg/kg, from about 0.1 to about 10 mg/kg, from about 1 to about 8 mg/kg, from about 1.5 to about 10 mg/kg, from about 2 to about 12 mg/kg, from about 2 to about 10 mg/kg, from about 3 to about 10 mg/kg, from about 4 to about 10 mg/kg, from about 4 to about 12 mg/kg, or from about 5 to about 12 mg/kg. Optionally, the TIMP-T1D is administered in a dose of about 0.01 mg/kg, about 0.02 mg/kg, about 0.05 mg/kg, about 0.1 mg/kg, about 0.25 mg/kg, about 0.5 mg/kg, about 1.0 mg/kg, about 2.0 mg/kg, about 4.0 mg/kg, about 6 mg/kg, about 8.0 mg/kg, about 10 mg/kg, or about 12 mg/kg. Alternatively, TIMP-T1D is administered at a dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In another embodiment, TIMP-T1D is administered at a concentration of between 0.005 mg/mL and 50 mg/mL inclusive of all values and ranges between these values, optionally about 0.05 mg/mL, 0.1 mg/mL, 0.5 mg/mL, 1 mg/mL, 2 mg/mL, 3 mg/mL, 3.25 mg/mL, 3.5 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

It is contemplated that the TIMP-T1D is administered in a single dose or in multiple doses. In various embodiments, TIMP-T1D is administered once weekly, once every two weeks, once every three weeks, once every 4 weeks, once every two months, once every three months, once every 6 months, once per year, once every two years, once every three years, once every four years, once every five years, once every six years, once every seven years, once every eight years, once every nine years, or once every ten years. In certain embodiments, TIMP-T1D is administered in two doses one-week apart.

In various embodiments, the method further comprises administering at least one booster dose of TIMP-T1D. In various embodiments, a booster dose of TIMP-T1D is administered in a single dose or in multiple doses following the loading TIMP-T1D administration. In various embodiments, the booster dose is administered once weekly, once every two weeks, once every three weeks, once every 4 weeks, once every two months, once every three months, once every 4 months, once every 5 months, once every 6 months, or once per year. In various embodiments, TIMP-T1D is administered in two loading doses one-week apart followed by a booster dose administered as a single dose once every three months. In various embodiments, one, two, three, four or five booster doses are administered to a subject.

In various embodiments, the booster dose of TIMP-T1D is administered at a dose of 0.01 to 12 mg/kg inclusive of all values and ranges between these values. In various embodiments, the booster dose of TIMP-T1D is administered at a dose from about 0.01 to about 12 mg/kg, from about 0.05 to about 10 mg/kg, from about 0.01 to about 5 mg/kg, from about 0.1 to about 10 mg/kg, from about 1 to about 8 mg/kg, from about 1.5 to about 10 mg/kg, from about 2 to about 12 mg/kg, from about 2 to about 10 mg/kg, from about 3 to about 10 mg/kg, from about 4 to about 10 mg/kg, from about 4 to about 12 mg/kg, or from about 5 to about 12 mg/kg, or about 0.01 mg/kg, about 0.05 mg/kg, about 0.1 mg/kg, about 0.25 mg/kg, about 0.5 mg/kg, about 1.0 mg/kg, about 2.0 mg/kg, about 3.0 mg/kg, about 4.0 mg/kg, about 5 mg/kg, about 6 mg/kg, about 8.0 mg/kg, about 10 mg/kg, or about 12 mg/kg. In various embodiments, the booster dose of TIMP-T1D is administered at a dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, TIMP-T1D is administered intravenously, subcutaneously, intramuscularly, intraperitoneally, intranasally, intra-lymphatically, or orally. In various embodiments, TIMP-T1D is administered intravenously, subcutaneously, intramuscularly, intraperitoneally, intranasally, inhaled, intra-lymphatically, or orally. It is contemplated that if TIMP-T1D is given intravenously, it can be via intravenous infusion lasting about 1, 2, 3, 4, 5, 6, 7, or 8 hours.

TIMP-T1D therapy is contemplated to relieve, lessen or ameliorate one or more signs or symptoms of T1D. Symptoms of T1D include, but are not limited to increased or frequent urination, increased thirst, increased hunger, extreme fatigue, blurry vision, loss of muscle, cuts or bruises that are slow to heal, coronary artery disease with chest pain (angina), heart attack, stroke, atherosclerosis, high blood pressure, nerve damage (neuropathy), kidney damage (nephropathy), diabetic retinopathy, diabetic ketoacidosis, foot damage, increased bacterial and viral infection in skin and mouth, gum disease, pregnancy complications like miscarriage, stillbirth and birth defects, exogenous insulin use, hypoglycemic events, elevated HbA1c, deteriorating β-cell function, declining C-peptide levels, decreased time in glycemic target range of 70-110 mg/dL (3.9 to 6.1 mmol/L) glucose, decreased time in glycemic target range of 70-180 (3.9 to 10 mmol/L) i.e. hyperglycemia, and weight loss.

TIMP-T1D therapy is also contemplated to decrease, shorten or ameliorate the duration and severity of an autoimmune or inflammatory immune response to one or more T1D antigens in a subject. An autoimmune or inflammatory immune response includes a T cell response, B cell response, Th1 response, myeloid cell response, and/or an antibody response. In various embodiments, administration of TIMP-T1D induces immunologic anergy. In various embodiments, the administration results in apoptosis of macrophages or monocytes in the subject. In various embodiments, administration of TIMP-T1D decreases islet stress response, reduces ineffective regulation of effector T cells, reduces islet/beta-cell damage, decreases epigenetic scarring, decreases immune effector amplification, decreases metabolic dysfunction, or decreases immune infiltrate into islets. In various embodiments, administration of TIMP-T1D induces islet cell/beta-cell regeneration, increases beta-cell mass/number, induces Tr1 cells, increases T_{reg}:T_{eff} ratio, induces bystander tolerance, induces infectious tolerance, induces linked suppression, induces T-cell exhaustion, induces T-cell apoptosis, induces T-cell deletion, or induces genotype independent tolerance. In various embodiments, administration of TIMP-T1D induces retention/sequestration of effector T-cells, regulatory APCs, IL-10, IL-35, TGF-beta, CTLA-4 expression, ICOS expression, GITR expression, PD-L1/L2 expression, PD-1 expression, FasL marker, Foxp3, or PGE2. In various embodiments, administration of TIMP-T1D decreases IL-6, IL-12, IL-23, IL-1beta, TNF, type 1 interferons, CD80 expression, CD86 expression, CD40/40L signaling, or T-bet expression. In various embodiments, the efficacy of TIMP-T1D at relieving one or more symptoms of T1D and/or reducing the duration and severity of an inflammatory immune response to one or more T1D antigens is determined from the assay of one or more biological samples from the subject as described herein.

### Screening Methods

It is contemplated that induction of, and maintenance of immunological tolerance is monitored in a subject suffering from T1D, wherein the subject is treated, or about to undergo treatment, with antigen-specific tolerizing therapy comprising TIMPs encapsulating T1D antigens as described herein.

Methods of screening for cell types, cytokines or other measures of tolerance from a subject undergoing tolerizing therapy as described herein are known in the art. Methods of assessing tolerance are done using such techniques as flow cytometry, Mass Cytometry (CyTOF), ELISA, ELISPOT, in vitro or ex vivo cell stimulation assays (including, but not limited to, cell proliferation assays, macrophage stimulation assays), measuring autoantibodies or measuring immunoglobulin (Ig) serotype, e.g., by ImmunoCap assay.

In various embodiments, the immune tolerance status of a subject is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, enzymes, and/or combinations thereof relevant in the disease or disorder.

Cells assayed from the biological sample include immune cells, non-immune cells, and/or combinations thereof. Immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

One aspect of a subject's immune tolerance status, and immune signature, is determined by analyzing one or more proteins from one or more biological sample(s) from the subject. In various embodiments, the proteins are cytokines and/or chemokines. In various embodiments the proteins are cell signaling proteins. In various embodiments, the cytokines and chemokines are selected from the group consisting of IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, soluble CD14, and/or combinations thereof. In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1.

Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including ELISA and ImmunoCap.

One aspect of a subject's immune tolerance status, and immune signature, is determined by analyzing one or more cell-surface proteins from a biological sample(s). In various embodiments, the cell-surface proteins include CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2Rβ, IL-4R, IL-5Rα, CSF2Rβ, IL-6Rα, gp130, IL-7Rα, IL-9R, IL-10R, IL-12Rβ1, IL-12Rβ2, IL-13Rα1, IL-13Rα2, IL-15Rα, IL-21R, IL23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTBR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α, β, γ, δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, B7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In various embodiments, a subject's immune tolerance status, and immune signature, is determined by analyzing the subject's liver function. In various embodiments, the subject's liver function is determined by the assay of total cholesterol, triglyceride, LDL-cholesterol, aspartate aminotransferase (AST), alanine aminotransferase (ALT), gamma glutamyl transferase (GGT), alkaline phosphatase (ALP), albumin, total protein, total bilirubin, globulin, creatine kinase (CK), and lactate dehydrogenase (LDH).

One aspect of a subject's immune tolerance status, and immune signature, is determined by analyzing one or more metabolites from the biological sample(s). In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), kynurenine, 3-hydroxy kynurenine, lipoxin A4, and lipoxin B4. In various embodiments, examples of anti-inflammatory metabolites include 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine.

A list of human metabolites that can be assayed from a biological sample can be found in the literature including in (Psychogios et al., 2011), (Wishart et al., HMDB: the Human Metabolome Database. Nucleic Acids Res. 2007 January; 35 (Database issue): D521-6, 2007), and the Human Metabalome Database (HMDB) and are incorporated herein by reference.

In certain embodiments, the subject's tolerance status is determined by analyzing nucleic acids from the biological sample(s). In various embodiments, the nucleic acids are DNA and/or RNA, including, but not limited to, single stranded DNA, double stranded DNA, mRNA, rRNA, tRNA, siRNA, miRNA, long non-coding RNAs (long ncRNAs, lncRNA), non-coding RNA (ncRNA), and mitochondrial RNA. In various embodiments, the miRNA is selected from the group consisting of miR-21, miR-34, miR-155, miR-146, miR-200, miR-429, miR-122, miR-451, miR-29, miR-7, miR-124, miR-23, miR-98, miR-590, miR-26, miR-101, miR-326, miR-15, miR-31, miR-149, miR-150, miR-9, miR-30, miR-25, miR-126, miR-144, miR-16, miR-192, miR-194, miR-27, miR-152, miR-148, miR-339, miR-425, miR-103a-3p, miR-155-5p, miR-200a-3p, miR-210-3p, hsa-miR-1-3p, hsa-miR-200b-3p, hsa-miR-9-5p, and hsa-miR-1200, miR-204-5P, miR-497, miR-21-3p, miR-424, miR-150-5p, miR-342, miR-491, miR-507, miR-517a-3p, miR-517B, miR-4691, miR-448, miR-370, miR-1236, miR-1267, miR-555, miR-93, miR-146a, miR-181, miR-30a-5p, miR-181a, miR-24, miR-210, miR-27a, miR-29a, miR-27b, miR-200a, miR-26a, miR-454-3p, miR-144-5p, miR-345-5p, miR-125a-3p, miR-502-3p, miR-500a-5p, miR-324-5p, miR-140-5p, miR-192-5p, miR-331-3p, miR-377-3p, miR-221-3p, miR-182-5p, miR-103a-2-5p, miR-183-5p, let-7e-5p, miR-18a-5p, miR-324-3p, miR-1468, miR-214-5p, miR-23b-3p, miR-720, miR-636, miR-630, miR-490-5p, miR-154-3p, miR-675-3p, miR-100-5p, miR-639, hsa-miR-146a-5p, hsa-miR-194-5p, hsa-miR-197-3p, hsa-miR-301a-3p, miR-197-3p, miR-122-5p, miR-125b-5p, miR-136-5p, miR-34a-5p, miR-342-3p, miR-320b, miR-28-5p, miR-151a-3p, miR-181a-5p, miR-151a-5p, miR-423-5p, miR-199a-3p, miR-126-3p, miR-652-3p, miR-148b-3p, miR-27b-3p, miR-107, miR-22-3p, miR-590-5p, let-7c-5p, miR-30d-5p, miR-106b-5p, miR-142-3p, miR-17-5p, let-7g-5p, miR-32-5p, miR-22-5p, miR-16-2-3p, miR-93-5p, miR-140-3p, miR-19a-3p, miR-19b-3p, miR-16-5p, miR-30e-5p, miR-363-3p, miR-222-3p, miR-144-3p, miR-24-3p, miR-1225-5p, miR-320c, miR-224, miR-148a, miR-26 miR-375, miR-302d-3p, miR-378e, miR-570-3p, miR-574-5p, miR-579, or miR-25-3p.

In various embodiments, the subject's immune tolerance status is determined by assaying gene expression from the biological sample(s). In various embodiments, the immune tolerance status is determined by assaying gene expression associated with immune function, an antibody, foreign body response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

The biological sample is optionally assayed after in vivo and/or ex vivo stimulation with one or more stimuli such as an antigen and one or more activating agents. It is contemplated that the T cells, B cells, and immunoglobulins used in the assay are antigen specific. Exemplary T cells include effector memory T cells, antigen-specific T cells, activated antigen-specific T cells, Th1 cells, Th17 cells, T follicular helper (TFH) cells, THO cells, or other antigen-specific T cells. B cells include effector B cells, memory B cells, plasma cells, and Breg cells. In certain embodiments, T cells are identified based on the expression of proteins described in Table 3.

TABLE 3

| Cell Type | Expression Marker |
|---|---|
| LEUKOCYTES | LIVE+, FSC, SSC |
| CD4 | LEUKOCYTES > CD3+ > CD8-CD4+ |
| T NAÏVE | CD4 > CCR7HI, CD45RA+ |
| CM | CD4 > CCR7HI, CD45RALOW |
| EM | CD4 > CCR7LO, CD45RALOW |
| T MEMORY (TMEM) | CD4 > CCR7HI/LOW, CD45RO+ |
| T1D SPECIFIC (DRUG | TMEM > CD154+, CD69+ |
| SUBSTANCE) | |
| T1D SPECIFIC 2 (DS2) | CD4 > CD154+, CD69+ |
| ACTIVATED DS | DS > CD38+ |
| T CELLS | |
| ACTIVATED DS2 T CELLS | DS2 > CD38+ |
| TH1 | DS > CXCR5−, CXCR3+, CCR6- |
| TH17 | DS > CXCR5−, CXCR3−, CCR6+ |
| TH2 | DS > CXCR5−, CXCR3−, CCR6-, CCR4+ |
| TFH | DS > CXCR5+ |
| TID SPECIFIC 3 (DS3) | CD4 > CD154−, CD137+, CD25+ |
| TREG | CD4 > CD154−, CD137+, CD127−, CD25+, |
| | FOXP3+, HELIOS+ |
| NON SPECIFC TREG | CD4 > CD25+, CD127− |
| TH1/TH17 | DS > CXCR5−, CXCR3+, CCR6+ |
| TH0 | DS > CXCR5−, CXCR3−, CCR6−, CCR4- |
| CD8 | CD3+ > CD8+, CD4− |
| DS CD8 | CD8 > CD154−, CD137+, CD69+ |
| CD8 ACTIVATED DS | DS CD8 > CD38+ |
| EXHAUSTED DS | DS > TIGIT+, KLRG1+, PD1+ |

In various embodiments, the immune tolerance status of the subject is determined by obtaining one or more samples, e.g., whole blood, from the subject pre-dose of the first TIMP-T1D administration (Day 1), 7 days after administration of the second dose (Day 15), and then at Day 60, Day 76, Day 90, Day 120, Day 166, Day 180, Day 256, Day 270, Day 346, and Day 360. Whole blood can then be processed to isolate peripheral blood mononuclear cells (PBMCs), basophils, neutrophils, plasma, and serum for downstream analyses. Assay of cells isolated from one or more samples collected from the subject and analyzed using such methods as described below.

In various embodiments, the immune tolerance status of the subject determined prior to administration of TIMP-T1D serves as the baseline. In various embodiments, the subject's baseline is determined from the assay of one or more biological samples 1, 2, 3, 4, 5, 6, or 7 days prior to administration of TIMP-T1D. In various embodiments, the subject's baseline is determined from the assay of one or more biological samples 1, 2, 3, or 4 weeks prior to administration of TIMP-T1D. In various embodiments, the subject's baseline is determined from the assay of one or more biological samples 1, 2, 3, 4, 5, 6, 7, 8, 9, 10, 11, or 12 months prior to administration of TIMP-T1D.

In various embodiments, the immune tolerance status of the subject is determined after administration of TIMP-T1D. In various embodiments, the immune tolerance status of the subject is determined from the assay of one or more biological samples 1, 2, 3, 4, 5, 6, or 7 days after the administration of TIMP-T1D. In various embodiments, the subject's status is determined from the assay of one or more biological samples 1, 2, 3, or 4 weeks after the administration of TIMP-T1D. In various embodiments, the subject's status is determined from the assay of one or more biological samples 1, 2, 3, 4, 5, 6, 7, 8, 9, 10, 11, or 12 months after administration of TIMP-T1D. In various embodiments, the subject's status is determined from the assay of one or more biological samples 1, 2, 3, 4, 5, 6, 7, 8, 9, 10, 11, or 12 years after administration of TIMP-T1D. In various embodiments, the immune tolerance status of the subject determined after administration of TIMP-T1D is compared to the baseline. In various embodiments, the immune tolerance status of the subject determined after the administration of TIMP-T1D is compared to a placebo control.

The immune tolerance signature of a subject is generated using one or more of the following parameters assayed from one or more biological samples obtained from the subject and stimulated in vivo and/or ex vivo:
A. proportion of effector T cells in the total T cell population,
B. proportion of Treg cells in the total T cell population,
C. proportion of effector B cells in the total B cell population,
D. levels of specific IgG, and/or IgM,
E. levels of inflammatory cytokines and chemokines,
F. levels of anti-inflammatory cytokines and chemokines,
G. levels of liver enzymes,
H. levels of inflammatory metabolites, and
I. levels of anti-inflammatory metabolites.

The immune tolerance signature is indicative of maintenance of immune tolerance if 1, 2, 3, 4, 5, 6, 7, 8, or 9 parameters listed in (a)-(i) above indicate maintenance of immune tolerance. In various embodiments, the immune tolerance signature is indicative of maintenance of immune tolerance if at least 2/9 parameters listed in (a)-(i) indicate maintenance of immune tolerance. In various embodiments, the subject is determined to not require treatment with TIMPs if 1, 2, 3, 4, 5, 6, 7, 8, or 9 parameters listed in (a)-(i) above indicate maintenance of immune tolerance. In various embodiments, the subject is determined to not require treatment with TIMPs if at least 3/9 parameters listed in (a)-(i) above indicate maintenance of immune tolerance.

The immune tolerance signature of a subject generated using one or more parameters described herein indicates weakening and/or absence of immune tolerance prior to or after treatment with TIMP-T1D, if:
a. the proportion of effector T cells in the total T cell population is between 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), relative to the subject's baseline measurement and/or relative to a placebo control and/or
b. the proportion of Treg cells in the total T cell population is between 0.01-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values) relative to the subject's baseline measurement and/or relative to a placebo control, and/or
c. the proportion of effector B cells in the total B cell population is between 0.01%-100% (e.g about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), relative to the subject's baseline measurement and/or relative to a placebo control and/or
d. the levels of IgG and/or IgM are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control, and/or
e. levels of inflammatory cytokines/chemokines are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control, and/or
f. levels of anti-inflammatory cytokines and chemokines are decreased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a relative to the subject's baseline measurement and/or relative to a placebo control, and/or
g. levels of liver enzymes are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control, and/or
h. levels of inflammatory metabolites are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control, and/or
i. levels of anti-inflammatory metabolites are decreased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

The efficacy of TIMP-T1D at relieving one or more symptoms of T1D and/or reducing the duration and severity of an autoimmune or inflammatory immune response to T1D antigens is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, enzymes, and/or combinations thereof.

In various embodiments, the efficacy of TIMP-T1D at relieving one or more symptoms of T1D and/or reducing the duration and severity of an inflammatory immune response to T1D antigens is determined based on the assay of cells from one or more biological samples from the subject pre- and post-treatment with TIMP-T1D. In various embodiments, the cells are immune cells, non-immune cells, and/or combinations thereof. In various embodiments, immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

In various embodiments, the efficacy of TIMP-T1D at relieving one or more symptoms of T1D and/or reducing the duration and severity of an inflammatory immune response to T1D antigens is determined based on the assay of cell surface proteins from one or more biological samples from the subject pre- and post-treatment with TIMP-T1D. In various embodiments, the cell surface proteins are selected from the group consisting CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβEεRI, MHC-I, MHC-II, IL-IR, IL-2Rα, IL-2Rβ, IL-2Rγ, IL-3Rα, CSF2Rβ, IL-4R, IL-5Rα, CSF2Rβ, IL-6Rα, gp130, IL-7Rα, IL-9R, IL-10R, IL-12Rβ1, IL-12Rβ2, IL-13Rα1, IL-13Rα2, IL-15Rα, IL-21R, IL23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTBR, TIM-1, TIM-3, TIM-4, TIGIT, LAG-3, ICOS, ICOS-L, SLAM, SLAMF2, OX-40, OX-40L, GITR, GITRL, TLIA, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α, β, γ, ε, and, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In various embodiments, treatment with TIMP-T1D decreases the expression of inflammatory cell surface proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D increases the expression of anti-inflammatory cell surface proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, the efficacy of TIMP-T1D at relieving one or more symptoms of T1D and/or reducing the duration and severity of an inflammatory immune response to T1D antigens is determined based on the assay of proteins from one or more biological samples from the subject pre- and post-treatment with TIMP-T1D. In various embodiments, the proteins are cytokines and/or chemokines. In various embodiments the proteins are cell signaling proteins. In various embodiments, the cytokines and chemokines are selected from the group consisting of IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1a, CXCL4 (MIP-1B, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, and/or combinations thereof.

In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, caspase 1, caspase 2, caspase 3, caspase 4, caspase 5, caspase 6, caspase 7, caspase 8, Caspase 9, caspase 10, caspase 11, caspase 12, caspase 13, caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including ELISA and ImmunoCap.

In various embodiments, treatment with TIMP-T1D decreases the levels of inflammatory proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control. In various embodiments, treatment with TIMP-T1D increases the levels of anti-inflammatory proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, the efficacy of TIMP-T1D at relieving one or more symptoms of T1D and/or reducing the duration and severity of an inflammatory immune response to T1D antigens is determined based on the assay of metabolites from one or more biological samples from the subject pre- and post-treatment with TIMP-T1D. In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), kynurenine, 3-hydroxy kynurenine, lipoxin A4, and lipoxin B4. In various embodiments, examples of anti-inflammatory metabolites include 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine. In various embodiments, treatment with TIMP-T1D decreases the levels of inflammatory metabolites by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control. In various embodiments, treatment with TIMP-T1D increases the levels of anti-inflammatory metabolites by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, the efficacy of TIMP-T1D at relieving one or more symptoms of T1D and/or reducing the duration and severity of an inflammatory immune response to T1D antigens is determined based on the following assessments pre- and post-TIMP-T1D administration:
A. Change from baseline in area under the curve (AUC) of stimulated C-peptide response over the first 2 hours of a 4-hour MMTT.
B. Maintenance of C-peptide from baseline
C. Change from baseline in antigen specific T cells
D. Change from baseline in activated antigen specific T cells
E. Change from baseline in antigen specific Tregs
F. Change from baseline in HbA1c levels
G. Change from baseline in number of hypoglycemic events
H. Change from baseline in insulin use
I. Change from baseline in time in glycemic target range of 70-110 mg/dl (3.9-10.0 mmol/L)

In various embodiments, the efficacy of TIMP-T1D is determined based on the following one or more assessments pre- and post-TIMP-T1D administration. In various embodiments, the booster dose is administered when needed as determined based on the following one or more assessments pre- and post-TIMP-T1D administration:
A. Change in area under the curve (AUC) of stimulated C-peptide response over the first 2 hours of a 2 or 4-hour MMTT.
B. Maintenance of C-peptide levels.
C. Change in maximum C-peptide during MMTT
D. Change in C-peptide measured at 30, 60, 90, and 120 minutes during MMTT
E. Change in fasting C-peptide
F. Proinsulin-to-C-peptide ratio
G. Change in antigen specific T cells.
H. Change in activated antigen specific T cells.
I. Change in antigen specific Tregs.
J. Change in HbA1c levels.
K. Change in number of hypoglycemic or severe hypoglycemic events
L. Change in rate of hypoglycemic events
M. Change in exogenous insulin use/consumption.
N. Change in time in glycemic target range of 70-180 mg/dL (3.9-10.0 mmol/L)
O. Change in insulin-dose-adjusted HbA1c (IDAA1c)
P. Insulin sensitivity score (ISS)
Q. Change in glycemic variability/fluctuations
R. Proportion of patients with IDAA1c≤9
S. Proportion of patients with a stimulated maximum C-peptide level above 0.2 nmol/L (0.6 ng/ml) at 15 months.
T. Proportion of patients with a stimulated 90 min C-peptide level above 0.2 nmol/L (0.6 ng/ml) at 15 months.
U. Change in body weight and body mass index
V. Concentrations of serum autoantibodies (number, and titer).
W. Concentrations of serum autoantibody isotypes (IgG1, IgG2, IgG3, IgG4).
X. Secretion of cytokines IL-1, IL-2, IL-5, IL-13, IL-10, IL-17, interferon (IFN)γ, tumour necrosis factor (TNF)α by PBMCs upon stimulation with T1D-associated antigens
Y. Serum concentrations of cytokines IL-1, IL-2, IL-5, IL-13, IL-10, IL-17, IFNγ, TNFα
Z. Secretion of cytokines IL-1, IL-2, IL-5, IL-13, IL-10, IL-17, interferon (IFN)γ, tumour necrosis factor (TNF)α by PBMCs upon stimulation with anti-CD3 and anti-CD28.
AA. Proliferation of PBMCs upon stimulation with T1D-associated antigens
BB. Circulating methylated- and unmethylated-insulin DNA levels
CC. Circulating hormone levels (e.g. glucagon, incretins, adiponectin)

In various embodiments, treatment with TIMP-T1D stabilizes the AUC of stimulated C-peptide response over the first 2 hours of a 4-hour MMTT relative to the subject's baseline measurement and/or relative to a placebo control. In various embodiments, treatment with TIMP-T1D increases the AUC of stimulated C-peptide response over the first 2 hours of a 4-hour MMTT by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the C-peptide levels relative to the subject's baseline measurement and/or relative to a placebo control. In various embodiments, treatment with TIMP-T1D increases the C-peptide levels by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the antigen specific T cells relative to the subject's baseline measurement and/or relative to a placebo control. In various embodiments, treatment with TIMP-T1D decreases the antigen specific T cells by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the activated antigen specific T cells relative to the subject's baseline measurement and/or relative to a placebo control. In various embodiments, treatment with TIMP-T1D decreases the activated antigen specific T cells by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the antigen specific Treg cells relative to the subject's baseline measurement and/or relative to a placebo control. In various embodiments, treatment with TIMP-T1D increases the antigen specific Treg cells by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes HbA1c levels relative to the subject's baseline measurement and/or relative to a placebo control. In various embodiments, treatment with TIMP-T1D decreases HbA1c levels by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control. In various embodiments, treatment with TIMP-T1D will decrease absolute HbA1c levels to below 140 mg/dL (or 6.5% HbA1c). In various embodiments, treatment with TIMP-T1D will decrease absolute HbA1c levels to between 137 mg/dL (6.4% HbA1c) and 117 mg/dL (5.7% HbA1c). In various embodiments, treatment with TIMP-T1D will decrease absolute HbA1c levels to below 117 mg/dL (5.7% HbA1c).

In various embodiments, treatment with TIMP-T1D stabilizes hypoglycemic events relative to the subject's baseline measurement and/or relative to a placebo control. In various embodiments, treatment with TIMP-T1D decreases hypoglycemic events by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes exogenous insulin use relative to the subject's baseline measurement and/or relative to a placebo control. In various embodiments, treatment with TIMP-T1D decreases exogenous insulin use by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes time in glycemic target range of 70-180 mg/dL (3.9-10.0 mmol/L) relative to the subject's baseline measurement and/or relative to a placebo control. In various embodiments, treatment with TIMP-T1D increases time in glycemic target range of 70-180 mg/dL (3.9-10.0 mmol/L) by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control. In various embodiments, time in glycemic range is measured by continuous glucose monitoring.

In various embodiments, treatment with TIMP-T1D stabilizes time in glycemic target range of 70-110 mg/dL (3.9-6.1 mmol/L) relative to the subject's baseline measurement and/or relative to a placebo control. In various embodiments, treatment with TIMP-T1D increases time in glycemic target range of 70-180 mg/dL (3.9-6.1 mmol/L) by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control. In various embodiments, time in glycemic range is measured by continuous glucose monitoring.

In various embodiments, treatment with TIMP-T1D stabilizes the C-peptide level measured at 30, 60, 90, and 120 minutes during MMTT relative to the subject's baseline measurement and/or relative to a placebo control. In various embodiments, treatment with TIMP-T1D increases C-peptide level measured at 30, 60, 90, and 120 minutes during MMTT by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes fasting C-peptide relative to the subject's baseline measurement and/or relative to a placebo control. In various embodiments, treatment with TIMP-T1D increases the fasting C-peptide by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the Proinsulin-to-C-peptide ratio relative to the subject's baseline measurement and/or relative to a placebo control. In various embodiments, treatment with TIMP-T1D decreases the Proinsulin-to-C-peptide ratio by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the insulin-dose-adjusted HbA1c (IDAA1c) relative to the subject's baseline measurement and/or relative to a placebo control. In various embodiments, treatment with TIMP-T1D decreases the IDAA1c by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the Insulin Sensitivity Score (ISS) relative to the subject's baseline measurement and/or relative to a placebo control. In various embodiments, treatment with TIMP-T1D decreases the ISS by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes glycemic variability/fluctuations relative to the subject's baseline measurement and/or relative to a placebo control. In various embodiments, treatment with TIMP-T1D decreases glycemic variability/fluctuations by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes the proportion of patients with IDAA1c≤9 relative to a placebo control. In various embodiments, treatment with TIMP-T1D increases the proportion of patients with IDAA1c≤9 by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a placebo control.

In various embodiments, treatment with TIMP-T1D stabilizes the proportion of patients with a stimulated maximum C-peptide level above 0.2 nmol/L relative to a placebo control. In various embodiments, treatment with TIMP-T1D increases the proportion of patients with a stimulated maximum C-peptide level above 0.2 nmol/L by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a placebo control.

In various embodiments, treatment with TIMP-T1D stabilizes secretion of cytokines IL-1, IL-2, IL-5, IL-13, IL-10, IL-17, interferon (IFN)γ, tumor necrosis factor (TNF)α from PBMCs upon stimulation with T1D-associated antigens relative to the subject's baseline measurement and/or relative to a placebo control. In various embodiments, treatment with TIMP-T1D alters secretion of cytokines IL-1, IL-2, IL-5, IL-13, IL-10, IL-17, interferon (IFN)γ, tumor necrosis factor (TNF)α from PBMCs upon stimulation with T1D-associated antigen by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes serum concentrations of cytokines IL-1, IL-2, IL-5, IL-13, IL-10, IL-17, IFNγ, TNFα relative to the subject's baseline measurement and/or relative to a placebo control. In various embodiments, treatment with TIMP-T1D alters serum secretion of cytokines IL-1, IL-2, IL-5, IL-13, IL-10, IL-17, IFNγ, TNFα by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes secretion of cytokines IL-1, IL-2, IL-5, IL-13, IL-10, IL-17, interferon (IFN)γ, tumor necrosis factor (TNF)α from PBMCs upon stimulation with anti-CD3 and anti-CD28 relative to the subject's baseline measurement and/or relative to a placebo control. In various embodiments, treatment with TIMP-T1D alters secretion of cytokines IL-1, IL-2, IL-5, IL-13, IL-10, IL-17, interferon (IFN) γ, tumor necrosis factor (TNF) α from PBMCs upon stimulation with anti-CD3 and anti-CD28 by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes proliferation of PBMCs upon stimulation with T1D-associated antigens relative to the subject's baseline measurement and/or relative to a placebo control. In various embodiments, treatment with TIMP-T1D decreases proliferation of PBMCs upon stimulation with T1D-associated antigens 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes circulating methylated- or unmethylated-insulin DNA levels relative to the subject's baseline measurement and/or relative to a placebo control. In various embodiments, treatment with TIMP-T1D decreases circulating unmethylated-insulin DNA levels by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

In various embodiments, treatment with TIMP-T1D stabilizes circulating hormone levels (e.g. glucagon, incretins, adiponectin) relative to the subject's baseline measurement and/or relative to a placebo control. In various embodiments, treatment with TIMP-T1D alters circulating hormone levels (e.g. glucagon, incretins, adiponectin) by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a placebo control.

The efficacy of TIMP-T1D at improving one or more symptoms of T1D is determined from one or more of the following parameters assayed from one or more biological samples obtained from the subject:
A. proportion of effector T cells in the total T cell population,
B. proportion of Treg cells in the total T cell population,
C. proportion of effector B cells in the total B cell population,
D. levels of specific IgG, and/or IgM,
E. levels of inflammatory cytokines and chemokines,
F. levels of anti-inflammatory cytokines and chemokines,
G. levels of liver enzymes,
H. levels of inflammatory metabolites, and
I. levels of anti-inflammatory metabolites.

In various embodiments, the efficacy of TIMP-T1D at improving one or more symptoms of T1D is determined from the result of the assay of 1, 2, 3, 4, 5, 6, 7, 8, or 9 parameters listed in (a)-(i) above.

In various embodiments, efficacy of TIMP-T1D at improving one or more symptom of T1D and/or reducing the duration and severity of an inflammatory response to T1D antigens is determined based on the use of alternative T1D therapies. In various embodiments, administration of TIMP-T1D decreases the use of alternative T1D therapies. In various embodiments, administration of TIMP-T1D stabilizes the use of alternative T1D therapies. In various embodiments, the therapy is selected from the group consisting of insulin, insulin pumps, high blood pressure medication, aspirin, cholesterol-lowering drugs, artificial pancreas, anti-IL-21 antibody, B-lymphocyte depletion (e.g. rituximab), anti-CD3 antibody (e.g. teplizumab), adoptive transfer of Tregs (e.g. autologous Tregs, umbilical cord derived Tregs, polyclonal Tregs), antithymocyte globulin, sodium-glucose cotransporter-2 (SGLT2) inhibitors (e.g. dapagliflozin, sotagliflozin), stem cell mobilization (e.g. Plerixafor), β-cell transplant, islet transplant, stem cell transplant, incretins therapy (e.g. liraglutide). In various embodiments, the insulin is selected from the group consisting of insulin glargine, rapid acting insulin (e.g. insulin aspart, insulin lispro, insulin glulisine), short acting insulin (ACTRAPID®, HUMULIN S®, INSUMAN RAPID®), long acting insulin (LEVEMIR®, ABASAGLAR®, LANTUS®, TOUJEO®, TRESIBA®) or intermediate acting (INSULATARD®, INSUMAN BASAL®).

In various embodiments, administration of TIMP-T1D decreases use of alternative T1D therapies 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% relative to the subject's baseline measurement and/or relative to a placebo control.

In various embodiments, the use of alternative T1D therapies is decreased or eliminated 1, 2, 3, 4, 5, 6, or 7 days after administration of TIMP-T1D. In various embodiments, the use of alternative T1D therapies is decreased or eliminated 1, 2, 3, or 4 weeks after administration of TIMP-T1D. In various embodiments, the use of alternative T1D therapies is decreased or eliminated 1, 2, 3, 4, 5, 6, 7, 8, 9, 10, 11, or 12 months after administration of TIMP-T1D. In various embodiments, the use of alternative T1D therapies is decreased or eliminated 1, 2, 3, 4, 5, 6, 7, 8, 9, 10, 11, or 12 years after administration of TIMP-T1D.

Cells assayed from the biological sample include immune cells, non-immune cells, and/or combinations thereof. Immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

### Combination Therapy

Concurrent administration of two therapeutic agents does not require that the agents be administered at the same time or by the same route, as long as there is an overlap in the time period during which the agents are exerting their therapeutic effect. Prior, simultaneous or sequential administration is contemplated, as is administration on different days or weeks.

It is contemplated that the TIMP-T1D and combination therapeutic may be given concomitantly, or simultaneously, in the same formulation or separate formulation. It is further contemplated that the TIMP-T1D and combination therapeutic are administered in a separate formulation and administered concomitantly, with concomitantly referring to agents given within 30 minutes to 12 hours of each other.

In another aspect, a TIMP-T1D and combination therapeutic is administered prior to administration of the other composition. Prior administration refers to administration of TIMP-T1D and combination therapeutic within the range of one week prior to treatment with the other therapy, up to 30 minutes before administration of the other therapy.

In another aspect, a TIMP-T1D and T1D therapeutic is administered subsequent to administration of the other composition. Subsequent administration refers to administration of TIMP-T1D and T1D therapeutic within the range of one week after treatment with the other therapy, up to 30 minutes after administration of the other therapy.

It is further contemplated that other adjunct or alternative therapies may be administered, where appropriate.

In various embodiments, TIMP-T1D is administered alone or in combination with a T1D therapeutic. In various embodiments, the therapeutic is administered prior to, concomitantly, or simultaneously, with or subsequent to the administration of TIMP-T1D.

In various embodiments, the combination therapeutic is selected from the group consisting of a insulin, insulin pumps, high blood pressure medication, aspirin, cholesterol-lowering drugs, artificial pancreas, anti-IL-21 antibody, B-lymphocyte depletion (e.g. rituximab), anti-CD3 antibody (e.g. teplizumab), adoptive transfer of Tregs (e.g. autologous Tregs, umbilical cord derived Tregs, polyclonal Tregs), antithymocyte globulin, sodium-glucose cotransporter-2 (SGLT2) inhibitors (e.g. dapagliflozin, sotagliflozin), stem cell mobilization (e.g. Plerixafor), β-cell transplant, islet transplant, stem cell transplant, incretins therapy (e.g. liraglutide). In various embodiments, the insulin is selected from the group consisting of insulin glargine, rapid acting insulin (e.g. insulin aspart, insulin lispro, insulin glulisine), short acting insulin (ACTRAPID®, HUMULIN S®, INSUMAN RAPID®), long acting insulin (LEVEMIR®, ABASAGLAR®, LANTUS®, TOUJEO®, TRESIBA®) or intermediate acting (INSULATARD®, INSUMAN BASAL®).

In various embodiments, the combination therapeutic induces Tregs. In various embodiments, the combination therapeutic increases the frequency and/or the number Tregs. In various embodiments, the combination therapeutic is IL-2 therapy to induce Tregs. In various embodiments, the IL-2 therapy is low dose IL-2, IL-2 muteins engineered to expand Tregs, IL-2 variants engineered to expand Tregs, IL-2 molecules engineered to be selective for the high-affinity IL-2 receptor, PEGylated IL-2, IL-2 complexes, or IL-2/CD25 fusion proteins,

In various embodiments, the therapeutic is administered 1, 2, 3, 4, 5, 6, or 7 days prior to administration of TIMP-T1D. In various embodiments, the therapeutic is administered 1, 2, 3, or 4 weeks prior to administration of TIMP-T1D. In various embodiments, the therapeutic is administered 1, 2, 3, 4, 5, 6, 7, 8, 9, 10, 11, or 12 months prior to administration of TIMP-T1D. In various embodiments, the therapeutic is administered 1, 2, 3, 4, 5, 6, 7, 8, 9, 10, 11, or 12 years prior to administration of TIMP-T1D. In various embodiments, the therapeutic is administered 1, 2, 3, 4, 5, 6, or 7 days subsequent to administration of TIMP-T1D. In various embodiments, the therapeutic is administered 1, 2, 3, or 4 weeks subsequent to administration of TIMP-T1D. In various embodiments, the therapeutic is administered 1, 2, 3, 4, 5, 6, 7, 8, 9, 10, 11, or 12 months subsequent to administration of TIMP-T1D. In various embodiments, the therapeutic is administered 1, 2, 3, 4, 5, 6, 7, 8, 9, 10, 11, or 12 years subsequent to administration of TIMP-T1D.

### Pharmaceutical Formulations

Pharmaceutical compositions of the present disclosure containing the TIMP-T1D described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carbox-yvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant, and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP-T1D described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a TIMP alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

In a further embodiment, the disclosure provides an article of manufacture, or unit dose form, comprising: (a) a composition of matter comprising TIMP-T1D as described herein; (b) a container containing said composition; and (c) a label affixed to said container, or a package insert included in said container referring to the use of said TIMP-T1D in the treatment of T1D as described herein.

Additional aspects and details of the disclosure will be apparent from the following examples, which are intended to be illustrative rather than limiting.

### EXAMPLES

### Example 1: Phase 1B/2A Trial of TIMP-T1D in Patients with T1D

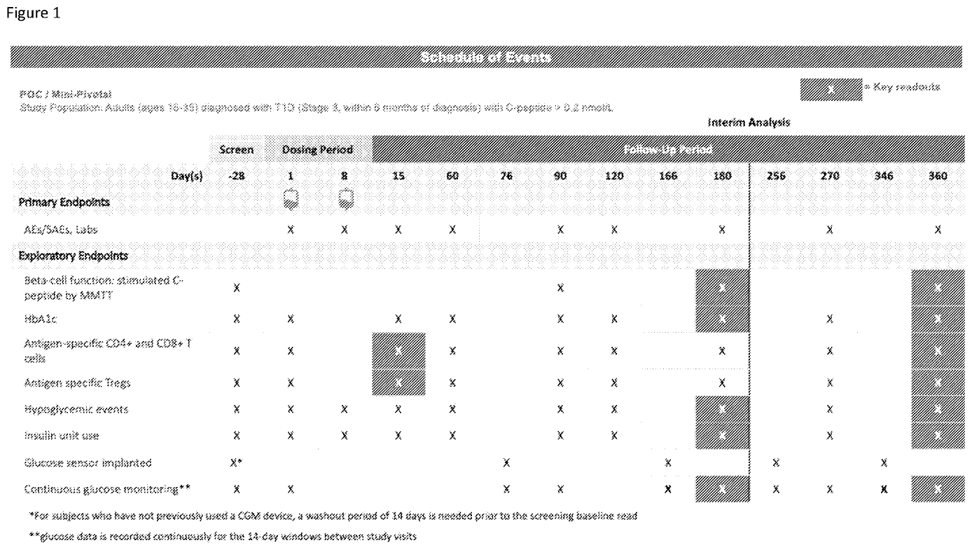

A phase 1b/2a double-blind, randomized, placebo-controlled study to assess the safety, tolerability, pharmacodynamics (PD), and efficacy of TIMP-T1D particles (CNP-103) will be carried out. CNP-103 consists of PLGA nanoparticles encapsulating T1D antigens comprising PPI, ZnT8, IGRP, and GAD65. CNP-103 particles have an average diameter of 400-800 nm and a negative zeta potential of between −30 mV and −80 mV. CNP-103 is supplied as a lyophilized formulation. CNP-103 is reconstituted in sterile water for injection and diluted in sterile saline (0.9% sodium chloride, USP) prior to administration. Subjects ages 18-35 with Stage 3 recent onset Type 1 Diabetes (T1D) will be screened up to 14 days prior to enrollment into the study. Note: If subjects are currently on a standard of care therapy, they will remain on their current standard of care therapy during the course of the study at the discretion of the Investigator regardless of treatment group (CNP-103 or Placebo). Screening will be completed per the Schedule of Events, (FIG. 1). Subjects who meet all inclusion and no exclusion criteria after completing the Screening Visit will be enrolled in the study. Subjects will then be randomized on Day 1 into the dose level Cohort open at the time in a 1:1 ratio to receive two separate administrations of intravenous CNP-103 or Placebo on Day 1 and Day 8. Study drug will be administered by IV infusion over approximately 3-4 hours using a graduated rate of infusion. Subjects will undergo medical observation in the clinic for acute AEs for 4 hours following infusion on Day 1 and Day 8. Subjects will be discharged after 4 hours if all scheduled assessments for the visit day have been completed, if vital signs (sitting or supine blood pressure, heart rate, and body temperature) measured 4 hours post infusion are within expected ranges for the subject and if no other health concerns are noted by the Investigator. In the post-dosing period, subjects will return to the clinic for immune safety labs, PD, measurements, hypoglycemic events and insulin unit use reporting, assessment of AEs, and medication changes per the Schedule of Events, (FIG. 1). On Screen Day, Day 90, Day 180 and Day 360 study visit, subjects will undergo a mixed meal tolerance test (MMTT). On Screen Day, Day 76, Day 166, Day 256, and Day 346 patients will have a glucose sensor implanted for continuous glucose monitoring. Glucose data (glycemic target) is read from the sensor On Screen Day, Day 1, Day 76, Day 90, Day 166, Day 180, Day 256, Day 270, Day 346 and Day 360. Subjects will return to the clinic for the end of study visit for an MMTT, collection of safety labs, PD measurements, hypoglycemic events and insulin unit use reporting, and final assessments of AEs and medication changes.

The study is planned to enroll up to 3 Cohorts (6 subjects per Cohort) at multiple ascending dose levels in the Dose Level Evaluation Phase followed by an Expansion Phase with a safe and tolerated dose identified in the Dose Level Evaluation Phase. Subjects will be randomized in a 1:1 ratio to receive either CNP-103 or Placebo (0.9% Sodium Chloride Injection USP) as a 200 mL intravenous infusion on Day 1 and Day 8. The planned dose levels are as follows:
Cohort 1:150 mg
Cohort 2:350 mg
Cohort 3:550 mg

CNP-103 will be administered using a graduated rate of infusion as follows:
20 mL/hr for first 15 min,
40 mL/hr, for next 15 min.
80 mL/hr for the remainder of the infusion.

Dosing of subjects within Cohort 1 will be separated by at least 48 hours. After all subjects in a dose Cohort have completed the Day 15 office visit (7 days post-second dose), the DMC will be convened to review all available safety data and determine whether it is acceptable to proceed to the next ascending dose Cohort, if an expansion of the Cohort is warranted, or if any other clinical recommendations should be made. After all subjects in a dose Cohort have completed Day 180, an interim analysis will be performed.

### Criteria for Inclusion

1. Subjects who are willing and able to provide Institutional Review Board (IRB) approved written informed consent and privacy language as per national regulations.
2. Men and non-pregnant, non-breast feeding women ages 18-35 years inclusive.
3. Diagnosis of T1D within 6 months prior to study enrollment according to ≥1 auto-antibody and at least 1 of the American Diabetes Association [ADA] criteria:
   A fasting plasma glucose (FPG) level ≥126 mg/dL (7.0 mmol/L),
   A random plasma glucose ≥200 mg/dL (11.1 mmol/L) in a patient with classic symptoms of hyperglycemia or hyperglycemic crisis
4. Patients with a peak stimulated C-peptide of >0.2 nmol/L at screen measured from a mixed meal tolerance test (MMTT).
5. Female subjects of non-childbearing potential (ex. Surgical sterilization, no menses for a year).
6. Women of child-bearing potential who have agreed not to become pregnant during the study, have a negative pregnancy test at Screening Visit and agree to one of the following:
   a. Use two highly effective forms of birth control starting at initial screening and continuing throughout the entire study to Day 360.
   b. Practice abstinence starting at initial screening and continuing throughout the entire study to Day 360.
7. Female subjects who agree to not breastfeed starting at initial screening and throughout the entire study to Day 360.
8. Female subjects who agree to not donate ova starting at initial screening and throughout the entire study to Day 360.
9. Male subject and with a spouse or partner of childbearing potential, who themselves and their spouse or partner agree to practice an effective form of birth control as discussed with the study doctor or study staff starting at screening and throughout the entire study to Day 360.

### Criteria for Exclusion

1. Subjects who have used the following medications:
   T cell depleting immunotherapy (ex. Teplizumab) within 12 months prior to the first dose
   T cell modifying immunotherapy (ex. Abatacept, Etanercept, Ustekinumab) within 6 months prior to the first dose.
   Exclusion of additional immunomodulation will be at the discretion of the medical monitor and study site investigator
2. Subjects who have any complicating medical issues that would interfere with blood drawing or monitoring.
3. Subjects who have have a Body Mass Index (BMI) that is less than the 5th percentile and greater than the 95th percentile for age and gender.
4. Subjects with a history of tuberculosis or positive PPD skin test.
5. Subjects who have received administration of vaccinations in the following time frame:
   Any live vaccine (other than intranasal Influenza) within 28 days prior to Screening;
   Any subunit vaccine within 14 days prior to Screening.
6. Any COVID-19 vaccine within 14 days prior to Screening. Subjects who have received the first dose of any COVID-19 vaccine may not screen for the study until 14 days following their last dose of the vaccine if applicable.
7. Known or suspected acute infection, including COVID-19 at the time of Screening or within 2 weeks prior to Screening. After confirmed recent COVID-19 infection, a negative polymerase chain reaction test will be required before randomization.
8. Any planned vaccination prior to Day 15.
9. Subjects who have used systemic steroids within 3 months prior to screening.
10. Subjects with laboratory test results at screening or prior to study dosing that are outside the normal limits and considered by the investigator to be clinically significant.
11. Subjects with positive test results for hepatitis B surface antigen (HBsAg), hepatitis C virus (HCV) antibody, or human immunodeficiency virus (HIV) antigen/antibody as determined at screening.
12. Subjects with a history of or currently active immune disorders other that T1D (including autoimmune disease) unless the condition, after discussion with the medical monitor, has been deemend to be acceptable for the subject's participation in this study.
13. Subjects with a history of or current active diseases requiring immunosuppressive drugs (including azathioprine, prednisone, prednisolone, budesonide, cyclosporine, tacrolimus, methotrexate, or mycophenolate mofetil) unless the condition, after discussion with the medical monitor, has been deemend to be acceptable for the subject's participation in this study.
14. Subjects with a clinical history of significant cardiovascular disease in the past 12 months.
15. Subjects with a complication or medical history of malignant tumor.
16. Subjects who, in the investigator's opinion, will be unable to adhere to study procedures.
17. Subjects who have received an investigational therapy other than CNP-103 within 28 days or 5 half-lives, whichever is longer, prior to screening.
18. Subjects with any known active condition which, in the investigator's opinion, makes the subject unsuitable for study participation.
19. Known sensitivity to any components of CNP-103.

Primary objectives of the study include assessing safety and tolerability of CNP-103.

### General Safety Assessments:

Frequency of Adverse Events (AEs) and Serious Adverse Events (SAEs), MedDRA 23.0 (CTCAE v. 5.0) or current.

### Immune Safety Assessments:

Serum Cytokines (IL-1β, TNF-α, IL-6, MCP-1, MIP-1α, IFN-γ, IL-4, IL-10)

Secondary or exploratory objectives of the study include assessing efficacy of CNP-103.
Change from baseline in area under the curve (AUC) of stimulated C-peptide response over the first 2 hours of a 4-hour MMTT between CNP-103 and placebo at Day 180 and Day 360
Maintenance of C-peptide from baseline between CNP-103 and placebo at Day 180 and Day 360
Change from baseline in antigen specific T cells between CNP-103 and placebo at Day 15
Change from baseline in activated antigen specific T cells between CNP-103 and placebo at Day 15
Change from baseline in antigen specific Tregs between CNP-103 and placebo at Day 15
Change from baseline in HbA1c levels between CNP-103 and placebo at Day 180 and Day 360
Change from baseline in number of hypoglycemic events between CNP-103 and placebo at Day 180 and Day 360
Change from baseline in insulin use between CNP-103 and placebo at Day 180 and Day 360
Change from baseline in time in glycemic target range of 70-180 mg/dL (3.9-10.0 mmol/L) [measured by continuous glucose monitoring] between CNP-103 and placebo at Day 180 and Day 360

### Example 2: Booster Dosing in Pre-Clinical Nod Mice Studies

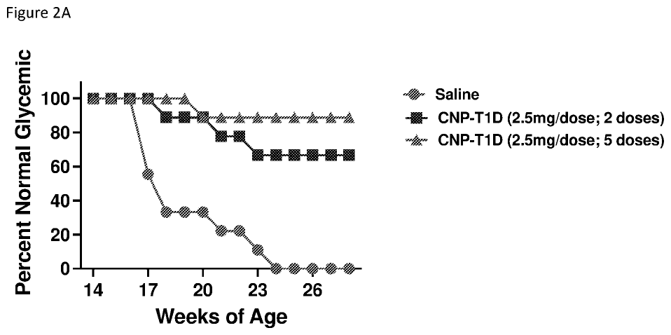

Pre-clinical dosing of CNP-T1D in NOD mice with booster doses. CNP-T1D comprises surface functionalized carboxylated PLGA nanoparticles, 400-800 nm size diameter, −80 mV to −30 mV encapsulating recombinant insulin, GAD65, and Chromogranin A. Dysglycemic NOD mice, with blood glucose levels of 150-250 mg/dL were divided into three groups. One group was treated with two loading doses of CNP-T1D at 16 weeks and 17 weeks. The second group was treated with two loading doses of CNP-T1D at 16 and 17 weeks followed by booster doses every 4 weeks (at 21 weeks, 25 weeks, 29 weeks) followed by a final take-down. Third group was the control group treated with saline. Treatment of mice with two doses of CNP-T1D significantly protected mice from the development of T1D with enhanced protection observed with the inclusion of booster doses when both were compared to saline (FIG. 2A).

Dysglycemic NOD mice, with blood glucose levels of 150-250 mg/dL were divided into three groups. The first group was treated with two loading doses of 2.5 mg CNP-T1D at 16 weeks and 17 weeks. The second group was treated with two loading doses of 2.5 mg CNP-T1D at 16 and 17 weeks followed by 2.5 mg booster doses every 4 weeks (at 21 weeks, 25 weeks, 29 weeks) followed by a final take-down at 30 weeks. The third group was the control group treated with saline.

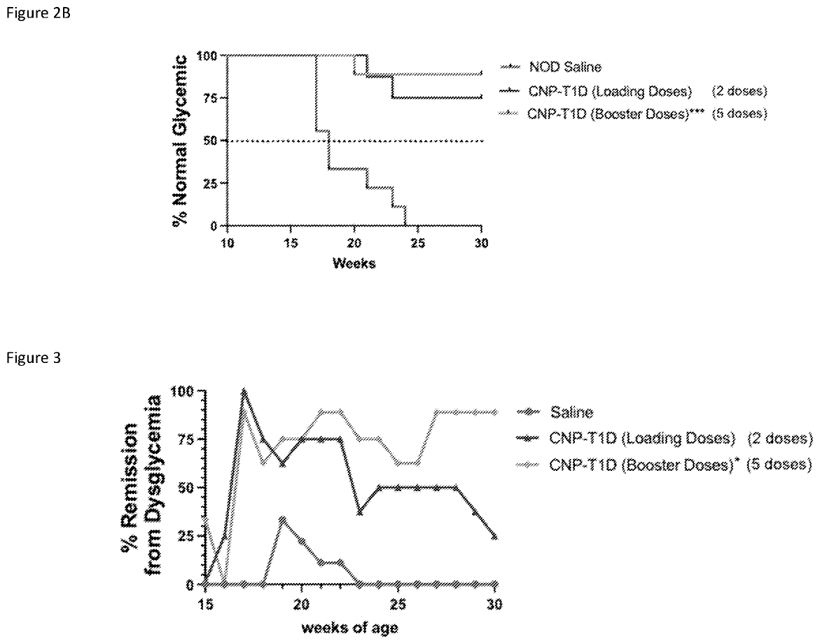

Treatment of mice with either 2 or 5 doses of 2.5 mg CNP-T1D significantly improved protection from T1D progression in dysglycemic NOD mice compared with control saline treated NOD mice. (*** p<0.0005) (FIG. 2A and FIG. 2B). However, reversal of dysglycemia i.e. reduction of blood glucose levels to normoglycemic levels (<150 mg/dL) was more pronounced in the NOD mice that received the booster dosing schedule with 5 doses compared to mice that received 2 loading doses. Comparison between 2.5 mg doses of CNP-T1D, 2 doses versus 5 doses, is significant. (*p=0.0183) (FIG. 3). These results indicates that TIMP-T1D is effective in inducing tolerance in NOD mice and treating symptoms of type1 diabetes.

### Example 3: Phase 1B/2A Trial of TIMP-T1D in Patients with T1D with 3 Doses

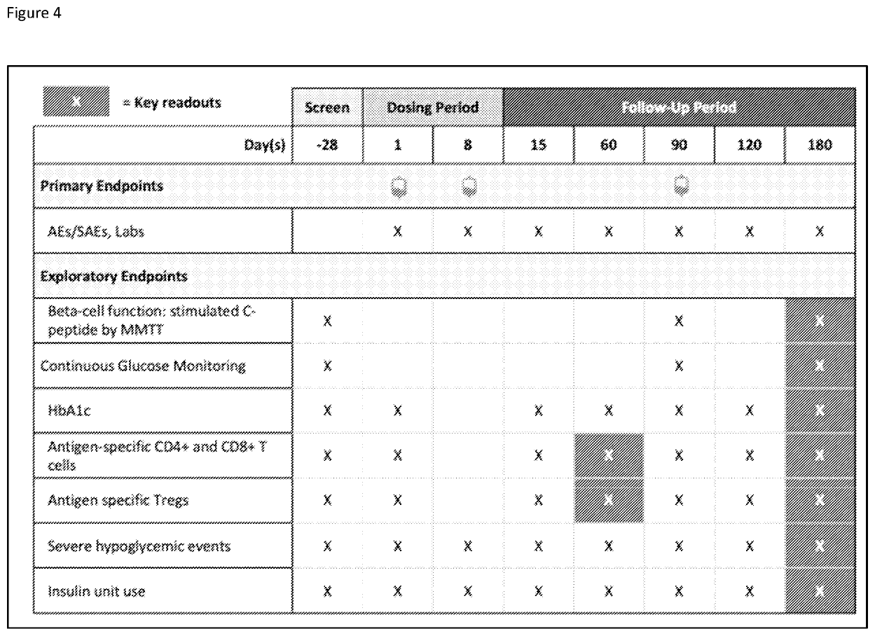

This study is a Phase 1b/2a First-in-Human (FIH) clinical trial to assess the safety, tolerability, pharmacodynamics (PD), and efficacy of multiple ascending doses of CNP-103. The study consists of an Escalation and Expansion Phase with 28 days for screening and 180 study days. Once the study is complete, all subjects will have the opportunity to roll-over into a long-term follow-up study. CNP-103 consists of PLGA (50:50 acid-end group) nanoparticles encapsulating T1D antigens comprising PPI, ZnT8, IGRP, and GAD65. CNP-103 particles have an average diameter of 400-800 nm and a negative zeta potential of between −30 mV and −80 mV. Subjects ages 18-35 with Stage 3 recent onset Type 1 Diabetes (T1D) will be screened up to 28 days prior to enrollment into the study. Note: If subjects are currently on a standard of care therapy, they will remain on their current standard of care therapy during the course of the study at the discretion of the Investigator regardless of treatment group (CNP-103 or Placebo). Screening will be completed per the Schedule of Events, (FIG. 4). Subjects who meet all inclusion and no exclusion criteria after completing the Screening Visit will be enrolled in the study. Subjects will then be randomized on Day 1 into the dose level Cohort open at the time in a 2:1 ratio to receive three separate administrations of intravenous CNP-103 or Placebo on Day 1, Day 8, and Day 90. Study drug will be administered by IV infusion over approximately 3-4 hours using a graduated rate of infusion. Subjects will undergo medical observation in the clinic for acute AEs for 4 hours following infusion on Day 1, Day 8, and Day 90. Subjects will be discharged after 4 hours if all scheduled assessments for the visit day have been completed, if vital signs (sitting or supine blood pressure, heart rate, and body temperature) measured 4 hours post-infusion are within expected ranges for the subject and if no other health concerns are noted by the Investigator. Subjects will return to the clinic for immune safety labs, PD, measurements, continuous glucose monitoring, hypoglycemic events and insulin unit use reporting, assessment of AEs, and medication changes per the Schedule of Events, (FIG. 4). On Screen Day, Day 90 and Day 180 study visit, subjects will undergo a mixed meal tolerance test (MMTT).

Subjects will return to the clinic for the end of study visit for an MMTT, collection of safety labs, PD measurements, continuous glucose monitoring, hypoglycemic events and insulin unit use reporting, and final assessments of AEs and medication changes.

The study is planned to enroll up to 3 Cohorts (6 subjects per Cohort) at multiple ascending dose levels in the Escalation Phase followed by an Expansion Phase with a safe and tolerated dose identified in the Escalation Phase. Subjects will be randomized in a 2:1 ratio to receive either CNP-103 or Placebo (0.9% Sodium Chloride Injection USP) as a 200 mL intravenous infusion on Day 1, Day 8, and Day 90. The planned dose levels are as follows:
Cohort 1:100 mg
Cohort 2:300 mg
Cohort 3:600 mg

CNP-103 will be administered using a graduated rate of infusion as follows:
20 mL/hr for first 15 min,
40 mL/hr, for next 15 min,
80 mL/hr for the remainder of the infusion.

Dosing of subjects within Cohort 1 will be separated by at least 48 hours. After all subjects in a dose Cohort have completed the Day 15 office visit (7 days post-second dose), the DMC will be convened to review all available safety data and determine whether it is acceptable to proceed to the next ascending dose Cohort, if an expansion of the Cohort is warranted, or if any other clinical recommendations should be made.

### Criteria for Inclusion

Subjects who are willing and able to provide Institutional Review Board (IRB) approved written informed consent and privacy language as per national regulations.
Men and non-pregnant, non-breast feeding women ages 18-35 years inclusive.
Documented diagnosis of T1D within 180 days prior to study enrollment according to ≥1 auto-antibody and at least 1 of the American Diabetes Association [ADA] criteria:
   A fasting plasma glucose (FPG) level ≥126 mg/dl (7.0 mmol/L),
   A random plasma glucose ≥200 mg/dL (11.1 mmol/L) in a patient with classic symptoms of hyperglycemia or hyperglycemic crisis
Patients with a peak stimulated C-peptide of >0.2 nmol/L at screen measured from a mixed meal tolerance test (MMTT).
For subjects on any medication used to treat the symptoms of T1D (ex. Corticosteroids), subjects must be on a stable dose for a minimum of 3 months prior to enrollment and must agree not to increase their dose through study Day 180 unless reviewed and approved by the medical monitor and the site investigator.
Female subjects of child-bearing potential who have agreed not to become pregnant during the study, have a negative pregnancy test at Screening Visit and agree to one of the following:
   Use two highly effective forms of birth control starting at initial screening and continuing throughout the entire study.
   Practice abstinence starting at initial screening and continuing throughout the entire study.
Female subjects who agree to not breastfeed starting at initial screening and throughout the entire study.
Female subjects who agree to not donate ova starting at initial screening and throughout the entire study.
Male subject and with a spouse or partner of childbearing potential, who themselves and their spouse or partner agree to practice an effective form of birth control as discussed with the study doctor or study staff starting at screening and throughout the entire study.

Primary objectives of the study include assessing safety and tolerability of CNP-103.

### General Safety Assessments:

Frequency of Adverse Events (AEs) and Serious Adverse Events (SAEs), MedDRA 23.0 (CTCAE v. 5.0) or current.

### Immune Safety Assessments:

Serum Cytokines (IL-1β, TNF-α, IL-6, MCP-1, MIP-1α, IFN-γ, IL-4, IL-10)

Secondary or exploratory objectives of the study include assessing efficacy of CNP-103.
Change from baseline in area under the curve (AUC) of stimulated C-peptide response over the first 2 hours of a 2-hour MMTT between CNP-103 and placebo at Day 180 and Day 360.
Maintenance of C-peptide from baseline between CNP-103 and placebo arms at Day 180 and Day 360.
Change from baseline in antigen specific T cells between CNP-103 and placebo at Day 60 and Day 180.
Change from baseline in activated antigen specific T cells between CNP-103 and placebo at Day 60 and Day 180.
Change from baseline in antigen specific Tregs between CNP-103 and placebo at Day 60 and Day 180.
Change from baseline in HbA1c levels between CNP-103 and placebo at Day 180 and Day
Change from baseline in number of severe hypoglycemic events between CNP-103 and placebo at Day 180 and Day 360
Change from baseline in insulin use between CNP-103 and placebo at Day 180 and Day 360
Change from baseline in time in glycemic target range of 70-180 mg/dL (3.9-10.0 mmol/L) [measured by continuous glucose monitoring] between CNP-103 and placebo at Day 180 and Day 360.

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

### REFERENCES

1. Gepts, W. Islet changes suggesting a possible immune aetiology of human diabetes mellitus. Acta Endocrinol. Suppl. 1976; 205, 95-106.
2. Gregory, G. A. et al. Glolab incidence, prevelance, and mortality of type 1 diabetes in 2021 with projection to 2040: a modelling study. The Lancet Diabetes & Endocrinology. 2022; 10: P741-760.

What is claimed is:

1. A method of treating Type 1 diabetes (T1D) in a subject comprising administering to the subject tolerizing immune modifying particles encapsulating one or more T1D associated antigens (TIMP-T1D), wherein TIMP-T1D is administered at a dose level between 1 mg and 800 mg.

2. The method of claim 1, wherein TIMP-T1D is administered at a fixed dose of 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

3. The method of claim 1 or claim 2, wherein TIMP-T1D is administered at dose level between about 0.01 mg/kg and 12 mg/kg.

4. The method of any one of claims 1-3, wherein the T1D associated antigen comprises insulin (preproinsulin (PPI)), tyrosine phosphatase like autoantigen or insulinoma antigen-2 (IA2; ICA512, PTPRN), IA-2β (Phogrin, PTPRN2), GAD65, GAD67, islet cell antigen-69 (ICA69), IGRP, ZnT8, Chromogranin A, 38 kDa granule antigen, peripherin, Islet amyloid peptide (ppIAPP), carboxypeptidase H/E, Heat shock protein 60 (HSP60), Heat shock protein 70 (HSP70) family, sulfatide, and vasostatin-1.

5. The method of any one of claims 1-4, wherein the T1D associated antigen is set out in Table 1.

6. The method of any one of claims 1-5, wherein the T1D associated antigen is selected from the group consisting of SEQ ID NO: 1 to SEQ ID NO: 17.

7. The method of any one of claims 1-6, wherein the T1D associated antigen comprises PPI, ZnT8, IGRP, and GAD65.

8. The method of claim 7, wherein the T1D associated antigen comprises SEQ ID NO: 1, SEQ ID NO: 2, SEQ ID NO: 3, and SEQ ID NO: 16.

9. The method of any one of the preceding claims, wherein the TIMP-T1D particles have an average diameter of between 100 nm and 1500 nm.

10. The method of any one of the preceding claims, wherein the TIMP-T1D particles have an average diameter of between 400 nm and 800 nm.

11. The method of any one of the preceding claims, wherein the TIMP-T1D particles have a negative zeta potential.

12. The method of any one of the preceding claims, wherein the particles have a negative zeta potential of between −30 mV and −100 mV.

13. The method of any one of the preceding claims, wherein TIMP-T1D is administered at a concentration of 0.05 mg/mL, 0.1 mg/mL, 0.5 mg/mL, 1 mg/mL, 2 mg/mL, 3 mg/mL, 3.25 mg/ml, 3.5 mg/ml, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

14. The method of any one of the preceding claims, wherein TIMP-T1D is administered at a dose level of 0.01 mg/kg, 0.05 mg/kg, 0.1 mg/kg, 0.25 mg/kg, 0.5 mg/kg, 1.0 mg/kg, 2.0 mg/kg, 4.0 mg/kg, 6 mg/kg, 8.0 mg/kg, 10 mg/kg, or 12 mg/kg.

15. The method of any one of the preceding claims, wherein TIMP-T1D is administered in a single dose or in multiple doses.

16. The method of any one of the preceding claims, wherein TIMP-T1D is administered once weekly, once every two weeks, once every three weeks, once every 4 weeks, once every two months, once every three months, once every 6 months, or once per year.

17. The method of any one of the preceding claims, wherein TIMP-T1D is administered in two doses one week apart.

18. The method of any one of the preceding claims, wherein TIMP-T1D is administered in two loading doses one-week apart followed by a booster dose administered as a single dose once every three months.

19. The method of any one of the preceding claims, wherein TIMP-T1D is administered intravenously, subcutaneously, intramuscular, intraperitoneally, intra-lymphatically, intranasally, inhaled, or orally.

20. The method of any one of the preceding claims, wherein administering TIMP-T1D to a subject decreases or ameliorates one or more symptoms of T1D.

21. The method of claim 20, wherein the one or more symptoms of T1D are selected from the group consisting of increased or frequent urination, increased thirst, increased hunger, extreme fatigue, blurry vision, loss of muscle, cuts or bruises that are slow to heal, coronary artery disease with chest pain, heart attack, stroke, atherosclerosis, high blood pressure, nerve damage, kidney damage, diabetic retinopathy, diabetic ketoacidosis, foot damage, increased bacterial and viral infection in skin and mouth, gum disease, pregnancy complications like miscarriage, stillbirth and birth defects, exogenous insulin use, hypoglycemic events, elevated HbA1c, deteriorating β-cell function, declining C-peptide levels, decreased time in glycemic target range of 70-110 mg/dL (3.9 to 6.1 mmol/L) glucose i.e. hyperglycemia and weight loss.

22. The method of any one of the preceding claims, wherein administering TIMP-T1D in a subject decreases duration and/or severity of an autoimmune or inflammatory immune response to T1D antigens.

23. The method of claim 22, wherein the autoimmune or inflammatory immune response is a T cell, B cell, or a myeloid cell response.

24. The method of claim 22 or 23, wherein the inflammatory immune response is assayed from one or more biological samples obtained from the subject.

25. The method of claim 24, wherein the biological sample is selected from the group consisting of whole-blood, peripheral blood, peripheral blood mononuclear cells (PBMCs), serum, plasma, urine, cerebrospinal fluid (CSF), stool, a tissue biopsy, and/or a bone-marrow biopsy.

26. The method of any one of claims 1-25, wherein administration of TIMP-T1D induces immunologic anergy.

27. The method of any one of claims 1-25, wherein administration of TIMP-T1D results in apoptosis of macrophages or monocytes in the subject.

28. The method of any one of claims 1-25, wherein administration of TIMP-T1D decreases islet stress response, reduces ineffective regulation of effector T cells, reduces islet/beta-cell damage, decreases epigenetic scarring, decreases immune effector amplification, decreases metabolic dysfunction, or decreases immune infiltrate into islets.

29. The method of any one of claims 1-25, wherein administration of TIMP-T1D induces islet cell/beta-cell regeneration, increases beta-cell mass/number, induces Tr1 cells, increases Treg:Teff ratio, induces bystander tolerance, induces infectious tolerance, induces linked suppression, induces T-cell exhaustion, induces T-cell apoptosis, induces T-cell deletion, or induces genotype independent tolerance.

30. The method of any one of claims 1-25, wherein administration of TIMP-T1D induces retention/sequestration of effector T-cells, induces regulatory APCs, IL-10, IL-35, TGF-beta, CTLA-4 expression, ICOS expression, GITR expression, PD-L1/L2 expression, PD-1 expression, FasL marker, or PGE2.

31. The method of any one of claims 1-25, wherein administration of TIMP-T1D decreases IL-6, IL-12, IL-23, IL-1beta, TNF, type 1 interferons, CD80 expression, CD86 expression, CD40 expression, or T-bet expression.

32. The method of any one of the preceding claims, wherein administering TIMP-T1D in a subject decreases or stabilizes levels of activated antigen-specific T cells.

33. The method of claim 32, wherein administering TIMP-T1D in a subject decreases or stabilizes levels of activated antigen-specific CD4+ and/or CD8+ T cells.

34. The method of claim 32 or 33, wherein administering TIMP-T1D decreases the levels of activated antigen-specific T cells by 5%-100% or by 2-100-fold relative to the subject's baseline measurement and/or relative to a placebo control.

35. The method of any one of the preceding claims, wherein administering TIMP-T1D in a subject decreases or stabilizes levels of antigen-specific T cells.

36. The method of claim 35, wherein administering TIMP-T1D in a subject decreases or stabilizes levels of antigen-specific CD4+ and/or CD8+ T cells.

37. The method of claim 35 or 36, wherein administering TIMP-T1D decreases the levels of antigen-specific T cells by 5%-100% or by 2-100-fold relative to the subject's baseline measurement and/or relative to a placebo control.

38. The method of any one of the preceding claims, wherein administering TIMP-T1D in a subject increases levels of antigen-specific Treg cells.

39. The method of claim 38, wherein administering TIMP-T1D increases the levels of antigen-specific Tregs cells by 5%-100% or by 2-100-fold relative to the subject's baseline measurement and/or relative to a placebo control.

40. The method of any one of the preceding claims, wherein administering TIMP-T1D in a subject stabilizes or increases the area under the curve (AUC) of stimulated C-peptide response.

41. The method of claim 40, wherein administering TIMP-T1D increases the area under the curve (AUC) of stimulated C-peptide response by 5%-100% or by 2-100-fold relative to the subject's baseline measurement and/or relative to a placebo control.

42. The method of any one of the preceding claims, wherein administering TIMP-T1D in a subject stabilizes or increases C-peptide levels.

43. The method of claim 42, wherein administering TIMP-T1D increases the C-peptide levels by 5%-100% or by about 2-100-fold relative to the subject's baseline measurement and/or relative to a placebo control.

44. The method of any one of the preceding claims, wherein administering TIMP-T1D in a subject stabilizes or decreases HbA1c levels.

45. The method of claim 44, wherein administering TIMP-T1D decreases HbA1c levels to below 140 mg/dL (or 6.5% HbA1c), to between 137 mg/dL (6.4% HbA1c) and 117 mg/dL (5.7% HbA1c), or below 117 mg/dL (5.7% HbA1c).

46. The method of claim 44 or 45, wherein administering TIMP-T1D decreases HbA1c levels by 5%-100% or by about 2-100-fold relative to the subject's baseline measurement and/or relative to a placebo control.

47. The method of any one of the preceding claims, wherein administering TIMP-T1D in a subject stabilizes or decreases hypoglycemic events.

48. The method of claim 47, wherein administering TIMP-T1D in a subject decreases hypoglycemic events by 5%-100% or by 2-100-fold.

49. The method of any one of the preceding claims, wherein administering TIMP-T1D in a subject stabilizes or decreases insulin use.

50. The method of claim 49, wherein administering TIMP-T1D in a subject decreases insulin use by 5%-100% or by 2-100-fold.

51. The method of any one of the preceding claims, wherein administering TIMP-T1D in a subject stabilizes or increases time in glycemic target range of 70-110 mg/dL (3.9-6.110.0 mmol/L).

52. The method of claim 51, wherein administering TIMP-T1D in a subject increases time in glycemic target range of 70-110 mg/dL (3.9-6.110.0 mmol/L) by 5%-100% or by 2-100-fold.

53. The method of claim 51 or 52, wherein time in glycemic range is measured by continuous glucose monitoring.

54. The method of any one of the preceding claims, wherein administering TIMP-T1D stabilizes or decreases the use of alternative therapies.

55. The method of claim 54, wherein the therapy is selected from the group consisting of insulin, insulin pumps, high blood pressure medication, aspirin, cholesterol-lowering drugs, artificial pancreas, anti-IL-21 antibody, B-lymphocyte depletion, anti-CD3 antibody, adoptive transfer of Tregs, anti-thymocyte globulin, sodium-glucose cotransporter-2 (SGLT2) inhibitors, stem cell mobilization, β-cell transplant, islet transplant, stem cell transplant, incretins therapy.

56. The method of claim 54 or 55, wherein administration of TIMP-T1D decreases use of alternative T1D therapies in the subject by 1%-100%.

57. The method of any one of the preceding claims, wherein TIMP-T1D is administered in combination with a T1D therapeutic.

58. The method of claim 57, wherein the combination therapeutic is selected from the group consisting of insulin, insulin pumps, high blood pressure medication, aspirin, cholesterol-lowering drugs, artificial pancreas, anti-IL-21 antibody, B-lymphocyte depletion, anti-CD3 antibody, adoptive transfer of Tregs, antithymocyte globulin, sodium-glucose cotransporter-2 (SGLT2) inhibitors, stem cell mobilization, β-cell transplant, islet transplant, stem cell transplant, incretins therapy.

59. The method of claim 58, wherein the insulin is selected from the group consisting of insulin glargine, rapid acting insulin, short acting insulin, long acting insulin or intermediate acting insulin.

60. The method of claim 57, wherein the combination therapeutic increases the frequency and/or the number of Tregs.

61. The method of claim 60, wherein the combination therapeutic is IL-2 therapy to induce Tregs.

62. The method of claim 61, wherein the combination therapeutic is low dose IL-2, IL-2 muteins engineered to expand Tregs, IL-2 variants engineered to expand Tregs, IL-2 molecules engineered to be selective for the high-affinity IL-2 receptor, PEGylated IL-2, IL-2 complexes, or IL-2/CD25 fusion proteins.

63. The method of any one of claims 57-62, wherein the therapeutic is administered prior to, concomitantly with or subsequent to the administration of TIMP-T1D.

64. The method of any one of the preceding claims, wherein administering a booster dose of TIMP-T1D is determined based on one or more of the following assessments: Change in area under the curve (AUC) of stimulated C-peptide response over the first 2 hours of a 2 or 4-hour MMTT, Maintenance of C-peptide levels, Change in maximum C-peptide during MMTT, Change in C-peptide measured at 30, 60, 90, and 120 minutes during MMTT, Change in fasting C-peptide, Proinsulin-to-C-peptide ratio, Change in antigen specific T cells, Change in activated antigen specific T cells, Change in antigen specific Tregs, Change in HbA1c levels, Change in number of hypoglycemic or severe hypoglycemic events, Change in rate of hypoglycemic events, Change in exogenous insulin use/consumption, Change in time in glycemic target range of 70-180 mg/dL (3.9-10.0 mmol/L), Change in time in glycemic target range of 70-110 mg/dL (3.9-6.1 mmol/L), Change in insulin-dose-adjusted HbA1c (IDAA1c), Insulin sensitivity score (ISS), Change in glycemic variability/fluctuations, Proportion of patients with IDAA1c≤9, Proportion of patients with a stimulated maximum C-peptide level above 0.2 nmol/L (0.6 ng/ml) at 15 months, Proportion of patients with a stimulated 90 min C-peptide level above 0.2 nmol/L (0.6 ng/ml) at 15 months, Change in body weight and body mass index, Concentrations of serum autoantibodies, Concentrations of serum autoantibody isotypes (IgG1, IgG2, IgG3, IgG4, Secretion of cytokines IL-1, IL-2, IL-5, IL-13, IL-10, IL-17, interferon (IFN)γ, tumor necrosis factor (TNF)α by PBMCs upon stimulation with T1D-associated antigens, Serum concentrations of cytokines IL-1, IL-2, IL-5, IL-13, IL-10, IL-17, IFNγ, TNFα, Secretion of cytokines IL-1, IL-2, IL-5, IL-13, IL-10, IL-17, IFNγ, TNFα by PBMCs upon stimulation with anti-CD3 and anti-CD28, Proliferation of PBMCs upon stimulation with T1D-associated antigens, Circulating methylated- and unmethylated-insulin DNA levels, or Circulating hormone levels.

65. The method of any one of the preceding claims, wherein the subject has recent-onset T1D.

66. The method of any one of the preceding claims, wherein the subject has Stage 3 T1D.

67. The method of any one of the preceding claims, wherein the subject has ≥1 auto antibody specific for pancreatic β-cell proteins, autoantibodies to GAD65, IA-2, ZnT8, insulin, or islet cell cytoplasmic protein autoantibodies, an autoantibody level <50,000 IU/mL, a fasting plasma glucose (FPG) level ≥126 mg/dL (7.0 mmol/L), a random plasma glucose ≥200 mg/dL (11.1 mmol/L) with classic symptoms of hyperglycemia or hyperglycemic crisis, a peak stimulated C-peptide of ≥0.2 nmol/L during a mixed meal tolerance test, a fasting C-peptide level of ≥0.12 nmol/L (≥0.36 ng/ml), a fasting C-peptide level ≥0.12-≤1.5 nmol/L (≥0.36-≤4.5 ng/mL), possesses HbA1c levels ≥5.6%, possesses HbA1c levels about ≤12%, is on a stable insulin dose or insulin dosing regimen between 1 week and 1 month, has at least 20% of beta-cell function prior to administration of the first dose, is between about 7 and 75 years of age, has an HLA genotype selected from the group consisting of HLA-DQ, HLA-DR, HLA-DP, or HLA-B, has an HLA genotype DR3, DR4, or DR3-DR4.

68. A composition comprising tolerizing immune modifying particles encapsulating one or more T1D associated antigens (TIMP-T1D) for use in treating type 1 diabetes (T1D) in a subject, wherein the TIMP-T1D is administered at a dose level between 1 mg and 800 mg.

69. Use of a composition comprising tolerizing immune modifying particles encapsulating one or more T1D associated antigens (TIMP-T1D) in the preparation of a medicament for treating type 1 diabetes in a subject, wherein TIMP-T1D is administered at a dose level between 1 mg and 800 mg.

70. The composition or use of claim 68 or 69, wherein TIMP-T1D is administered at dose level between about 0.01 mg/kg and 12 mg/kg.

71. The composition or use of any one of claims 68-70, wherein the T1D associated antigen comprises insulin (preproinsulin (PPI)), tyrosine phosphatase like autoantigen or insulinoma antigen-2 (IA2; ICA512, PTPRN), IA-2β (Phogrin, PTPRN2), GAD65, GAD67, islet cell antigen-69 (ICA69), IGRP, ZnT8, Chromogranin A, 38 kDa granule antigen, peripherin, Islet amyloid peptide (ppIAPP), carboxypeptidase H/E, Heat shock protein 60 (HSP60), Heat shock protein 70 (HSP70) family, sulfatide, and vasostatin-1.

72. The composition or use of any one of claims 68-71, wherein the T1D associated antigen is selected from the group consisting of SEQ ID NO: 1 to SEQ ID NO: 17.

73. The composition or use of any one of claims 68-72, wherein the T1D associated antigen comprises PPI, ZnT8, IGRP, and GAD65.

74. The composition or use of claim 73, wherein the T1D associated antigen comprises SEQ ID NO: 1, SEQ ID NO: 2, SEQ ID NO: 3, and SEQ ID NO: 16.

75. The composition or use of any one of claims 68-74, wherein the TIMP-T1D particles have an average diameter of between 100 nm and 1500 nm.

76. The composition or use of any one of claims 68-75, wherein the TIMP-T1D particles have an average diameter of between 400 nm and 800 nm.

77. The composition or use of any one of claims 68-76, wherein the TIMP-T1D particles have a negative zeta potential.

78. The composition or use of any one of claims 68-77, wherein the particles have a negative zeta potential of between −30 mV and −100 mV.

79. The composition or use of any one of claims 69-78, wherein the T1D associated antigen comprises PPI, ZnT8, IGRP, and GAD65.


SEQUENCE LISTING (17 sequences)

SEQ ID NO: 1 - protein, 110 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 499 aa, synthetic construct
MNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 3 - protein, 355 aa, synthetic construct
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 4 - protein, 369 aa, synthetic construct
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 5 - protein, 979 aa, synthetic construct
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 6 - protein, 1015 aa, synthetic construct
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 7 - protein, 594 aa, synthetic construct
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 8 - protein, 483 aa, synthetic construct
MSGHKCSYPWDLQDRYAQDKSVVNKMQQKYWETKQAFIKATGKKEDEHVVASDADLDAKLELFHSIQRTCLDLSKAIVLYQKRICFLSQEENELGKFLRSQGFQDKTRAGKMMQATGKALCFSSQQRLALRNPLCRFHQEVETFRHRAISDTWLTVNRMEQCRTEYRGALLWMKDVSQELDPDLYKQMEKFRKVQTQVRLAKKNFDKLKMDVCQKVDLLGASRCNLLSHMLATYQTTLLHFWEKTSHTMAAIHESFKGYQPYEFTTLKSLQDPMKKLVEKEEKKKINQQESTDAAVQEPSQLISLEEENQRKESSSFKTEDGKSILSALDKGSTHTACSGPIDELLDMKSEEGACLGPVAGTPEPEGADKDDLLLLSEIFNASSLEEGEFSKEWAAVFGDGQVKEPVPTMALGEPDPKAQTGSGFLPSQLLDQNMKDLQASLQEPAKAASDLTAWFSLFADLDPLSNPDAVGKTDKEHELLNA

SEQ ID NO: 9 - protein, 457 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 10 - protein, 306 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKGRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 11 - protein, 470 aa, synthetic construct
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEVVTESQKEQRSELDKSSAHSY

SEQ ID NO: 12 - protein, 346 aa, synthetic construct
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESEGWLLEKSVPETWKAFLESVKKLGKGNQVEAEGAGAGQAPEAG

SEQ ID NO: 13 - protein, 89 aa, synthetic construct
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 14 - protein, 476 aa, synthetic construct
MAGRGGSALLALCGALAACGWLLGAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 15 - protein, 573 aa, synthetic construct
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 16 - protein, 368 aa, synthetic construct
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPC

SEQ ID NO: 17 - protein, 640 aa, synthetic construct
AKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD